(19) United States
(12) Patent Application Publication
(10) Pub. No.: US 2023/0255927 A1
(43) Pub. Date: Aug. 17, 2023

(54) MONENSIN LEVELS FOR MODERN DAIRY DIET

(71) Applicant: ELANCO TIERGESUNDHEIT AG, Basel (CH)

(72) Inventors: Sara KVIDERA, Greenfield, IN (US); Gerald MECHOR, Greenfield, IN (US)

(21) Appl. No.: 18/109,410

(22) Filed: Feb. 14, 2023

Related U.S. Application Data
(60) Provisional application No. 63/309,992, filed on Feb. 14, 2022.
(60) Provisional application No. 63/366,592, filed on Jun. 17, 2022.

(51) Int. Cl. A61K 31/351; A61P 15/14; A61P 3/00; A23K 50/10; A23K 20/195; A23K 20/121

(52) U.S. Cl. CPC A61K 31/351; A61P 15/14; A61P 3/00; A23K 50/10; A23K 20/195; A23K 20/121

(57) ABSTRACT

The present disclosure provides formulations and methods for administration of dietary monensin to be fed to ruminants, including cattle. As described herein, monensin can provide beneficial effects of increasing dietary monensin concentration on milk yield and milk component yield, including milk fat yield, milk fat percentage, milk protein yield, and milk protein percentage.

### CROSS-REFERENCE TO RELATED APPLICATIONS

This application claims the benefit under 35 USC § 119(e) of U.S. Provisional Application Ser. No. 63/309,992, filed on Feb. 14, 2022 and U.S. Provisional Application Ser. No. 63/366,592, filed on Jun. 17, 2022, the entire disclosures of both which are incorporated herein by reference.

### BACKGROUND AND SUMMARY OF THE INVENTION

Monensin is a polyether having antibiotic properties that is widely used in ruminant animal feeds. For instance, monensin (e.g., RUMENSIN®, Elanco Animal Health, Greenfield, Ind.) is known to increase feed efficiency in ruminants such as cattle. Although monensin can be included in ruminant feed at broad ranges (e.g., 11 g/ton to 22 g/ton), the effects of specific doses of monensin on cows are unknown. For instance, monensin doses fed to lactating cows could have desirable effects on milk components and milk component yield but these specific doses are currently unknown.

Accordingly, the present disclosure provides formulations and methods for administration of dietary monensin to be fed to ruminants. As described herein, monensin can provide beneficial effects of increasing dietary monensin concentration on milk yield and milk component yield, including milk fat yield, milk fat percentage, milk protein yield, and milk protein percentage. Further, monensin can provide a beneficial milk fatty acid (FA) profile (e.g., de novo, mixed, and preformed FAs), energy corrected milk (ECM), dry matter intake (DMI), energy corrected milk per unit of dry matter intake (ECM/DMI; also known as feed efficiency), body weight change, and dietary energy in ruminants.

In one aspect, as provided by the present disclosure, particular amounts of monensin fed to ruminants (e.g., 11 g/ton and 14.5 g/ton) unexpectedly demonstrated a statistically significant increase in milk production efficiency (as measured by energy corrected milk, ECM, divided by dry matter intake, DMI) and fatty acid profile. Advantageously, these monensin doses did not negatively impact milk fat percentages.

Various aspects of the disclosure are described more fully below with reference to the accompanying Appendix, which forms a part hereof, and which shows specific example aspects. However, different aspects of the disclosure may be implemented in many ways and should not be construed as limited to the aspects set forth herein. The following detailed description is, therefore, not to be taken in a limiting sense.

### BRIEF DESCRIPTION OF THE DRAWINGS

FIG. 1A shows a graphical representation of the effects of dietary monensin concentration on milk yield. Each bar from left to right represents data from Control, R11, R14.5, and R18, respectively. FIG. 1B shows a graphical representation of the effects of dietary monensin concentration on milk fat percentage. Each bar from left to right represents data from Control, R11, R14.5, and R18, respectively. FIG. 1C shows a graphical representation of the effects of dietary monensin concentration on milk fat yield. Each bar from left to right represents data from Control, R11, R14.5, and R18, respectively.

FIG. 2A shows a graphical representation of the effects of dietary monensin concentration on milk protein percentage. Each bar from left to right represents data from Control, R11, R14.5, and R18, respectively. FIG. 2B shows a graphical representation of the effects of dietary monensin concentration on milk protein yield.

FIG. 2C shows a graphical representation of the effects of dietary monensin concentration on Energy Corrected Milk (ECM). Each bar from left to right represents data from Control, R11, R14.5, and R18, respectively.

FIG. 3A shows a graphical representation of the effects of dietary monensin concentration on dry matter intake (DMI). Each bar from left to right represents data from Control, R11, R14.5, and R18, respectively. FIG. 3B shows a graphical representation of the effects of dietary monensin concentration on energy corrected milk per unit of dry matter intake (ECM/DMI). Each bar from left to right represents data from Control, R11, R14.5, and R18, respectively.

FIG. 4A shows a graphical representation of the effects of dietary monensin concentration on body weight change. Each bar from left to right represents data from Control, R11, R14.5, and R18, respectively. FIG. 4B shows a graphical representation of the effects of dietary monensin concentration on initial and final body weights. Each bar grouping from left to right represents data from Control, R11, R14.5, and R18, respectively. In each bar group, the unshaded bars represent initial body weights, and the shaded bars represent final body weights. FIG. 4C shows a graphical representation of the effects of dietary monensin concentration on estimated dietary energy. Each bar from left to right represents data from Control, R11, R14.5, and R18, respectively.

FIG. 5 shows a graphical representation of the effects of dietary monensin concentration on the composition of de novo production (left), mixed production (center), and preformed production (right) of fatty acids in milk. Each bar grouping from left to right represents data from Control, R11, R14.5, and R18, respectively.

FIG. 6A shows a graphical representation of dietary monensin intake. Each bar from left to right represents data from Control, R11, R14.5, and R18, respectively. FIG. 6B shows a graphical representation of the effects of dietary monensin concentration on dry matter intake (DMI). Each bar from left to right represents data from Control, R11, R14.5, and R18, respectively.

FIG. 7 shows a graphical representation of the effects of dietary monensin concentration on milk yield (left), energy-corrected milk (ECM, center), and fat-corrected milk (FCM, right). Each bar grouping from left to right represents data from Control, R11, R14.5, and R18, respectively.

FIG. 8A shows a graphical representation of the effects of dietary monensin concentration on fat and protein yield. Each bar from left to right represents data from Control, R11, R14.5, and R18, respectively. FIG. 8B shows a graphical representation of the effects of dietary monensin concentration on fat and protein content. Each bar from left to right represents data from Control, R11, R14.5, and R18, respectively.

FIG. 9 shows a graphical representation of the effects of dietary monensin concentration on milk urea nitrogen (MUN, left) and plasma urea nitrogen (PUN, right). Each bar grouping from left to right represents data from Control, R11, R14.5, and R18, respectively.

FIG. 10 shows a graphical representation of the effects of dietary monensin concentration on milk production efficiency (left), energy-corrected milk (ECM, center), and fat-corrected milk (FCM, right). Each bar grouping from left to right represents data from Control, R11, R14.5, and R18, respectively.

FIG. 11 shows a graphical representation of the effects of dietary monensin concentration on the composition of de novo production (left), mixed production (center), and preformed production (right) of milk fatty acids. Each bar grouping from left to right represents data from Control, R11, R14.5, and R18, respectively.

FIG. 12 shows a graphical representation of the effects of dietary monensin concentration on the composition of de novo production (left), mixed production (center), and preformed production (right) of milk fatty acid yields. Each bar grouping from left to right represents data from Control, R11, R14.5, and R18, respectively.

FIG. 13A shows a graphical representation of the effects of dietary monensin concentration fatty acid unsaturation. FIG. 13B shows a graphical representation of the effects of dietary monensin concentration fatty acid mean chain length. Each bar from left to right represents data from Control, R11, R14.5, and R18, respectively.

FIG. 14A shows a graphical representation of the effects of dietary monensin concentration on body weight. FIG. 14B shows a graphical representation of the effects of dietary monensin concentration on body weight change. FIG. 14C shows a graphical representation of the effects of dietary monensin concentration on body condition score (BCS). Each bar from left to right represents data from Control, R11, R14.5, and R18, respectively.

FIG. 15 shows a graphical representation of the effects of dietary monensin concentrations on milk production efficiency in mid-lactation cows by energy corrected milk per unit of dry matter intake (ECM/DMI). Each bar grouping from left to right represents data from Control, R11, R14.5, and R18, respectively.

FIG. 16 shows a graphical representation of the effects of dietary monensin concentrations on milk fat content percentages. Each bar grouping from left to right represents data from Control, R11, R14.5, and R18, respectively.

FIG. 17A shows a graphical representation of the effects of dietary monensin concentration on the composition of de novo production of milk fatty acid yields. Each bar grouping from left to right represents data from Control, R11, R14.5, and R18, respectively. FIG. 17B shows a graphical representation of the effects of dietary monensin concentration on the composition of mixed production of milk fatty acid yields. Each bar grouping from left to right represents data from Control, R11, R14.5, and R18, respectively. FIG. 17C shows a graphical representation of the effects of dietary monensin concentration on the composition of preformed production of milk fatty acid yields. Each bar grouping from left to right represents data from Control, R11, R14.5, and R18, respectively.

FIG. 18 shows a graph of dry matter intake for both locations (Wisconsin and New York) of lactating dairy cattle fed varying concentrations of monensin.

FIG. 19 shows a graph of body weight measurements for both locations (Wisconsin and New York) of lactating dairy cattle fed varying concentrations of monensin. †=Statistical tendency (0.05≤P≤0.10).

FIG. 20 shows a graph of sample day milk yield for both locations (Wisconsin and New York) of lactating dairy cattle fed varying concentrations of monensin.

FIG. 21 shows a graph of milk fat yield, calculated using sample day milk yield, for both locations (Wisconsin and New York) of lactating dairy cattle fed varying concentrations of monensin. *=Statistical significance (P<0.05); †=Statistical tendency (0.05≤P≤0.10).

FIG. 22 shows a graph of milk protein yield, calculated using sample day milk yield, for both locations (Wisconsin and New York) of lactating dairy cattle fed varying concentrations of monensin. †=Statistical tendency (0.05≤P≤0.10).

FIG. 23 shows a graph of milk lactose yield, calculated using sample day milk yield, for both locations (Wisconsin and New York) of lactating dairy cattle fed varying concentrations of monensin.

FIG. 24 shows a graph of milk urea nitrogen concentration for both locations (Wisconsin and New York) of lactating dairy cattle fed varying concentrations of monensin. *=Statistical significance (P<0.05).

FIG. 25 shows a graph of solids corrected milk yield, calculated using sample day milk yield, for both locations (Wisconsin and New York) of lactating dairy cattle fed varying concentrations of monensin. *=Statistical significance (P<0.05); †=Statistical tendency (0.05≤P≤0.10).

FIG. 26 shows a graph of energy corrected milk yield, calculated using sample day milk yield, for both locations (Wisconsin and New York) of lactating dairy cattle fed varying concentrations of monensin. *=Statistical significance (P<0.05); †=Statistical tendency (0.05≤P≤0.10).

FIG. 27 shows a graph of 4% fat corrected milk yield, calculated using sample day milk yield, for both locations (Wisconsin and New York) of lactating dairy cattle fed varying concentrations of monensin. *=Statistical significance (P<0.05); †=Statistical tendency (0.05≤P≤0.10).

FIG. 28 shows a graph of de novo production of milk fatty acids for both locations (Wisconsin and New York) of lactating dairy cattle fed varying concentrations of monensin. †=Statistical tendency (0.05≤P≤0.10).

FIG. 29 shows a graph of mixed production of milk fatty acids for both locations (Wisconsin and New York) of lactating dairy cattle fed varying concentrations of monensin.

FIG. 30 shows a graph of preformed production of milk fatty acids for both locations (Wisconsin and New York) of lactating dairy cattle fed varying concentrations of monensin. ∛=Statistical tendency (0.05≤P≤0.10).

FIG. 31 shows a graph of total production of milk fatty acids for both locations (Wisconsin and New York) of lactating dairy cattle fed varying concentrations of monensin.

### DETAILED DESCRIPTION

Various embodiments of the invention are described herein as follows. In an illustrative aspect, a dietary formulation for ruminants is provided. The dietary formulation comprises i) a feed composition and ii) an amount of monensin selected from a range of about 11 g/ton to about 16 g/ton of the feed composition.

In an embodiment, the ruminant is selected from the group consisting of cattle, goats, sheep, giraffes, American Bison, European bison, yaks, water buffalo, deer, camels, alpacas, llamas, wildebeest, antelope, pronghorn, and nilgai. In an embodiment, the ruminant is selected from the group consisting of cattle, buffalo, sheep, and goats. In an embodiment, the ruminant is a buffalo. In an embodiment, the ruminant is a sheep. In an embodiment, the ruminant is a goat. In an embodiment, the ruminant is a bovine. In an embodiment, the ruminant is a cow. In an embodiment, the cow is a lactating cow.

In an embodiment, the amount of monensin is selected from a range of about 11 g/ton to about 14.5 g/ton of the feed composition. In an embodiment, the amount of monensin is selected from a range of about 12 g/ton to about 16 g/ton of the feed composition. The amount of monensin in the described ranges can include various amounts, including 11.0 g/ton, 11.1 g/ton, 11.2 g/ton, 11.3 g/ton, 11.4 g/ton, 11.5 g/ton, 11.6 g/ton, 11.7 g/ton, 11.8 g/ton, 11.9 g/ton, 12.0 g/ton, 12.1 g/ton, 12.2 g/ton, 12.3 g/ton, 12.4 g/ton, 12.5 g/ton, 12.6 g/ton, 12.7 g/ton, 12.8 g/ton, 12.9 g/ton, 13.0 g/ton, 13.1 g/ton, 13.2 g/ton, 13.3 g/ton, 13.4 g/ton, 13.5 g/ton, 13.6 g/ton, 13.7 g/ton, 13.8 g/ton, 13.9 g/ton, 14.0 g/ton, 14.1 g/ton, 14.2 g/ton, 14.3 g/ton, 14.4 g/ton, 14.5 g/ton, 14.6 g/ton, 14.7 g/ton, 14.8 g/ton, 14.9 g/ton, 15.0 g/ton, 15.1 g/ton, 15.2 g/ton, 15.3 g/ton, 15.4 g/ton, 15.5 g/ton, 15.6 g/ton, 15.7 g/ton, 15.8 g/ton, 15.9 g/ton, and 16.0 g/ton.

In an embodiment, the amount of monensin is about 11 g/ton of the feed composition. In an embodiment, the amount of monensin is about 14.5 g/ton of the feed composition.

In an embodiment, the feed composition comprises one or more of corn silage, alfalfa silage, ground corn, canola meal, soybean meal, and liquid whey. In an embodiment, the feed composition comprises one or more of corn silage, grass haylage, ground corn, soybean meal, citrus pulp, wheat middlings, dextrose, and blood meal.

In an embodiment, providing the dietary formulation to a ruminant results in an increase in milk production efficiency in the ruminant. In an embodiment, the amount of monensin is about 14.5 g/ton of the feed composition, wherein a feed efficiency (ECM/DMI) in the ruminant is increased by about 3.5%. In an embodiment, the feed efficiency is increased by a higher percentage than for a feed composition including an amount of monensin of about 11 g/ton or about 16 g/ton. In an embodiment, the amount of monensin is about 11 g/ton of the feed composition, wherein a feed efficiency in the ruminant is increased by about 3.0%. In an embodiment, the amount of monensin is about 11 g/ton of the feed composition, wherein a milk fat yield is quadratically increased in the ruminant by about 0.08 kg/d over a feed composition not including monensin. In an embodiment, the amount of monensin is about 14.5 g/ton of the feed composition, wherein energy corrected milk is increased in the ruminant by about 1.13 kg/day over a feed composition not including monensin. In an embodiment, the amount of monensin is about 11 g/ton of the feed composition, wherein energy corrected milk is quadratically increased in the ruminant by about 1.3 kg/d over a feed composition not including monensin. In an embodiment, the amount of monensin is about 11 g/ton of the feed composition, wherein energy corrected milk is increased in the ruminant by about 0.86 kg/day over a feed composition not including monensin.

In an illustrative aspect, a method of increasing a milk production efficiency in a ruminant is provided. The method comprises providing a dietary formulation to the ruminant, wherein the dietary formulation comprises a feed composition and an amount of monensin selected from a range of about 11 g/ton to about 16 g/ton of the feed composition; and wherein milk production efficiency in the ruminant is increased. Any of the dietary formulations as described herein can be utilized according to the method.

In an embodiment, the ruminant is selected from the group consisting of cattle, goats, sheep, giraffes, American Bison, European bison, yaks, water buffalo, deer, camels, alpacas, llamas, wildebeest, antelope, pronghorn, and nilgai. In an embodiment, the ruminant is selected from the group consisting of cattle, buffalo, sheep, and goats. In an embodiment, the ruminant is a buffalo. In an embodiment, the ruminant is a sheep. In an embodiment, the ruminant is a goat. In an embodiment, the ruminant is a bovine. In an embodiment, the ruminant is a cow. In an embodiment, the cow is a lactating cow.

In an embodiment, the amount of monensin is selected from a range of about 11 g/ton to about 14.5 g/ton of the feed composition. In an embodiment, the amount of monensin is selected from a range of about 12 g/ton to about 16 g/ton of the feed composition. The amount of monensin in the described ranges can include various amounts, including 11.0 g/ton, 11.1 g/ton, 11.2 g/ton, 11.3 g/ton, 11.4 g/ton, 11.5 g/ton, 11.6 g/ton, 11.7 g/ton, 11.8 g/ton, 11.9 g/ton, 12.0 g/ton, 12.1 g/ton, 12.2 g/ton, 12.3 g/ton, 12.4 g/ton, 12.5 g/ton, 12.6 g/ton, 12.7 g/ton, 12.8 g/ton, 12.9 g/ton, 13.0 g/ton, 13.1 g/ton, 13.2 g/ton, 13.3 g/ton, 13.4 g/ton, 13.5 g/ton, 13.6 g/ton, 13.7 g/ton, 13.8 g/ton, 13.9 g/ton, 14.0 g/ton, 14.1 g/ton, 14.2 g/ton, 14.3 g/ton, 14.4 g/ton, 14.5 g/ton, 14.6 g/ton, 14.7 g/ton, 14.8 g/ton, 14.9 g/ton, 15.0 g/ton, 15.1 g/ton, 15.2 g/ton, 15.3 g/ton, 15.4 g/ton, 15.5 g/ton, 15.6 g/ton, 15.7 g/ton, 15.8 g/ton, 15.9 g/ton, and 16.0 g/ton.

In an embodiment, the amount of monensin is about 11 g/ton of the feed composition. In an embodiment, the amount of monensin is about 14.5 g/ton of the feed composition.

In an embodiment, the feed composition comprises one or more of corn silage, alfalfa silage, ground corn, canola meal, soybean meal, and liquid whey. In an embodiment, the feed composition comprises one or more of corn silage, grass haylage, ground corn, soybean meal, citrus pulp, wheat middlings, dextrose, and blood meal.

In an embodiment, the increase is a statistically significant increase in milk production efficiency.

In an illustrative aspect, a method of improving a fatty acid profile in a ruminant is provided. The method comprises providing a dietary formulation to the ruminant, wherein the dietary formulation comprises a feed composition and an amount of monensin selected from a range of about 11 g/ton to about 16 g/ton of the feed composition. Any of the dietary formulations as described herein can be utilized according to the method.

In an embodiment, the ruminant is selected from the group consisting of cattle, goats, sheep, giraffes, American Bison, European bison, yaks, water buffalo, deer, camels, alpacas, llamas, wildebeest, antelope, pronghorn, and nilgai. In an embodiment, the ruminant is selected from the group consisting of cattle, buffalo, sheep, and goats. In an embodiment, the ruminant is a buffalo. In an embodiment, the ruminant is a sheep. In an embodiment, the ruminant is a goat. In an embodiment, the ruminant is a bovine. In an embodiment, the ruminant is a cow. In an embodiment, the cow is a lactating cow.

In an embodiment, the amount of monensin is selected from a range of about 11 g/ton to about 14.5 g/ton of the feed composition. In an embodiment, the amount of monensin is selected from a range of about 12 g/ton to about 16 g/ton of the feed composition. The amount of monensin in the described ranges can include various amounts, including 11.0 g/ton, 11.1 g/ton, 11.2 g/ton, 11.3 g/ton, 11.4 g/ton, 11.5 g/ton, 11.6 g/ton, 11.7 g/ton, 11.8 g/ton, 11.9 g/ton, 12.0 g/ton, 12.1 g/ton, 12.2 g/ton, 12.3 g/ton, 12.4 g/ton, 12.5 g/ton, 12.6 g/ton, 12.7 g/ton, 12.8 g/ton, 12.9 g/ton, 13.0 g/ton, 13.1 g/ton, 13.2 g/ton, 13.3 g/ton, 13.4 g/ton, 13.5 g/ton, 13.6 g/ton, 13.7 g/ton, 13.8 g/ton, 13.9 g/ton, 14.0 g/ton, 14.1 g/ton, 14.2 g/ton, 14.3 g/ton, 14.4 g/ton, 14.5 g/ton, 14.6 g/ton, 14.7 g/ton, 14.8 g/ton, 14.9 g/ton, 15.0 g/ton, 15.1 g/ton, 15.2 g/ton, 15.3 g/ton, 15.4 g/ton, 15.5 g/ton, 15.6 g/ton, 15.7 g/ton, 15.8 g/ton, 15.9 g/ton, and 16.0 g/ton.

In an embodiment, the amount of monensin is about 11 g/ton of the feed composition. In an embodiment, the amount of monensin is about 14.5 g/ton of the feed composition.

In an embodiment, the feed composition comprises one or more of corn silage, alfalfa silage, ground corn, canola meal, soybean meal, and liquid whey. In an embodiment, the feed composition comprises one or more of corn silage, grass haylage, ground corn, soybean meal, citrus pulp, wheat middlings, dextrose, and blood meal.

In an embodiment, de novo fatty acids in the milk of the ruminant are increased. In an embodiment, mixed fatty acids in the milk of the ruminant are increased.

In an illustrative aspect, a method of increasing a feed efficiency (ECM/DMI) in a ruminant is provided. The method comprises providing a dietary formulation to the ruminant, wherein the dietary formulation comprises a feed composition and an amount of monensin selected from a range of about 11 g/ton to about 16 g/ton of the feed composition. Any of the dietary formulations as described herein can be utilized according to the method. In an embodiment, the ruminant is selected from the group consisting of cattle, goats, sheep, giraffes, American Bison, European bison, yaks, water buffalo, deer, camels, alpacas, llamas, wildebeest, antelope, pronghorn, and nilgai. In an embodiment, the ruminant is selected from the group consisting of cattle, buffalo, sheep, and goats. In an embodiment, the ruminant is a buffalo. In an embodiment, the ruminant is a sheep. In an embodiment, the ruminant is a goat. In an embodiment, the ruminant is a bovine. In an embodiment, the ruminant is a cow. In an embodiment, the cow is a lactating cow.

In an embodiment, the amount of monensin is selected from a range of about 11 g/ton to about 14.5 g/ton of the feed composition. In an embodiment, the amount of monensin is selected from a range of about 12 g/ton to about 16 g/ton of the feed composition. The amount of monensin in the described ranges can include various amounts, including 11.0 g/ton, 11.1 g/ton, 11.2 g/ton, 11.3 g/ton, 11.4 g/ton, 11.5 g/ton, 11.6 g/ton, 11.7 g/ton, 11.8 g/ton, 11.9 g/ton, 12.0 g/ton, 12.1 g/ton, 12.2 g/ton, 12.3 g/ton, 12.4 g/ton, 12.5 g/ton, 12.6 g/ton, 12.7 g/ton, 12.8 g/ton, 12.9 g/ton, 13.0 g/ton, 13.1 g/ton, 13.2 g/ton, 13.3 g/ton, 13.4 g/ton, 13.5 g/ton, 13.6 g/ton, 13.7 g/ton, 13.8 g/ton, 13.9 g/ton, 14.0 g/ton, 14.1 g/ton, 14.2 g/ton, 14.3 g/ton, 14.4 g/ton, 14.5 g/ton, 14.6 g/ton, 14.7 g/ton, 14.8 g/ton, 14.9 g/ton, 15.0 g/ton, 15.1 g/ton, 15.2 g/ton, 15.3 g/ton, 15.4 g/ton, 15.5 g/ton, 15.6 g/ton, 15.7 g/ton, 15.8 g/ton, 15.9 g/ton, and 16.0 g/ton.

In an embodiment, the amount of monensin is about 11 g/ton of the feed composition. In an embodiment, the amount of monensin is about 14.5 g/ton of the feed composition.

In an embodiment, the feed composition comprises one or more of corn silage, alfalfa silage, ground corn, canola meal, soybean meal, and liquid whey. In an embodiment, the feed composition comprises one or more of corn silage, grass haylage, ground corn, soybean meal, citrus pulp, wheat middlings, dextrose, and blood meal.

In an embodiment, the feed efficiency is increased by about 3.5% compared to a ruminant that does not receive dietary monensin. In an embodiment, the feed efficiency is increased by about 3.0% compared to a ruminant that does not receive dietary monensin.

In an illustrative aspect, a method of increasing milk fat yield in a ruminant is provided. The method comprises providing a dietary formulation to the ruminant, wherein the dietary formulation comprises a feed composition and an amount of monensin selected from a range of about 11 g/ton to about 16 g/ton of the feed composition. Any of the dietary formulations as described herein can be utilized according to the method.

In an embodiment, the ruminant is selected from the group consisting of cattle, goats, sheep, giraffes, American Bison, European bison, yaks, water buffalo, deer, camels, alpacas, llamas, wildebeest, antelope, pronghorn, and nilgai. In an embodiment, the ruminant is selected from the group consisting of cattle, buffalo, sheep, and goats. In an embodiment, the ruminant is a buffalo. In an embodiment, the ruminant is a sheep. In an embodiment, the ruminant is a goat. In an embodiment, the ruminant is a bovine. In an embodiment, the ruminant is a cow. In an embodiment, the cow is a lactating cow.

In an embodiment, the amount of monensin is selected from a range of about 11 g/ton to about 14.5 g/ton of the feed composition. In an embodiment, the amount of monensin is selected from a range of about 12 g/ton to about 16 g/ton of the feed composition. The amount of monensin in the described ranges can include various amounts, including 11.0 g/ton, 11.1 g/ton, 11.2 g/ton, 11.3 g/ton, 11.4 g/ton, 11.5 g/ton, 11.6 g/ton, 11.7 g/ton, 11.8 g/ton, 11.9 g/ton, 12.0 g/ton, 12.1 g/ton, 12.2 g/ton, 12.3 g/ton, 12.4 g/ton, 12.5 g/ton, 12.6 g/ton, 12.7 g/ton, 12.8 g/ton, 12.9 g/ton, 13.0 g/ton, 13.1 g/ton, 13.2 g/ton, 13.3 g/ton, 13.4 g/ton, 13.5 g/ton, 13.6 g/ton, 13.7 g/ton, 13.8 g/ton, 13.9 g/ton, 14.0 g/ton, 14.1 g/ton, 14.2 g/ton, 14.3 g/ton, 14.4 g/ton, 14.5 g/ton, 14.6 g/ton, 14.7 g/ton, 14.8 g/ton, 14.9 g/ton, 15.0 g/ton, 15.1 g/ton, 15.2 g/ton, 15.3 g/ton, 15.4 g/ton, 15.5 g/ton, 15.6 g/ton, 15.7 g/ton, 15.8 g/ton, 15.9 g/ton, and 16.0 g/ton.

In an embodiment, the amount of monensin is about 11 g/ton of the feed composition. In an embodiment, the amount of monensin is about 14.5 g/ton of the feed composition.

In an embodiment, the feed composition comprises one or more of corn silage, alfalfa silage, ground corn, canola meal, soybean meal, and liquid whey. In an embodiment, the feed composition comprises one or more of corn silage, grass haylage, ground corn, soybean meal, citrus pulp, wheat middlings, dextrose, and blood meal.

In an embodiment, the milk fat yield is quadratically increased by about 0.08 kg/d compared to a ruminant that does not receive dietary monensin.

In an illustrative aspect, a method of increasing energy corrected milk in a ruminant is provided. The method comprises providing a dietary formulation to the ruminant, wherein the dietary formulation comprises a feed composition and an amount of monensin selected from a range of about 11 g/ton to about 16 g/ton of the feed composition. Any of the dietary formulations as described herein can be utilized according to the method.

In an embodiment, the ruminant is selected from the group consisting of cattle, goats, sheep, giraffes, American Bison, European bison, yaks, water buffalo, deer, camels, alpacas, llamas, wildebeest, antelope, pronghorn, and nilgai. In an embodiment, the ruminant is selected from the group consisting of cattle, buffalo, sheep, and goats. In an embodiment, the ruminant is a buffalo. In an embodiment, the ruminant is a sheep. In an embodiment, the ruminant is a goat. In an embodiment, the ruminant is a bovine. In an embodiment, the ruminant is a cow. In an embodiment, the cow is a lactating cow.

In an embodiment, the amount of monensin is selected from a range of about 11 g/ton to about 14.5 g/ton of the feed composition. In an embodiment, the amount of monensin is selected from a range of about 12 g/ton to about 16 g/ton of the feed composition. The amount of monensin in the described ranges can include various amounts, including 11.0 g/ton, 11.1 g/ton, 11.2 g/ton, 11.3 g/ton, 11.4 g/ton, 11.5 g/ton, 11.6 g/ton, 11.7 g/ton, 11.8 g/ton, 11.9 g/ton, 12.0 g/ton, 12.1 g/ton, 12.2 g/ton, 12.3 g/ton, 12.4 g/ton, 12.5 g/ton, 12.6 g/ton, 12.7 g/ton, 12.8 g/ton, 12.9 g/ton, 13.0 g/ton, 13.1 g/ton, 13.2 g/ton, 13.3 g/ton, 13.4 g/ton, 13.5 g/ton, 13.6 g/ton, 13.7 g/ton, 13.8 g/ton, 13.9 g/ton, 14.0 g/ton, 14.1 g/ton, 14.2 g/ton, 14.3 g/ton, 14.4 g/ton, 14.5 g/ton, 14.6 g/ton, 14.7 g/ton, 14.8 g/ton, 14.9 g/ton, 15.0 g/ton, 15.1 g/ton, 15.2 g/ton, 15.3 g/ton, 15.4 g/ton, 15.5 g/ton, 15.6 g/ton, 15.7 g/ton, 15.8 g/ton, 15.9 g/ton, and 16.0 g/ton.

In an embodiment, the amount of monensin is about 11 g/ton of the feed composition. In an embodiment, the amount of monensin is about 14.5 g/ton of the feed composition.

In an embodiment, the feed composition comprises one or more of corn silage, alfalfa silage, ground corn, canola meal, soybean meal, and liquid whey. In an embodiment, the feed composition comprises one or more of corn silage, grass haylage, ground corn, soybean meal, citrus pulp, wheat middlings, dextrose, and blood meal.

In an embodiment, the energy corrected milk is increased by about 1.13 kg/day compared to a ruminant that does not receive dietary monensin. In an embodiment, the energy corrected milk is quadratically increased by about 1.3 kg/d over a feed composition not including monensin.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

### Example 1

### Wisconsin Study

The instant example, referred to herein as the Wisconsin Study, provides evaluation of the effect of monensin (RUMENSIN®, Elanco Animal Health, Greenfield, Ind.) administration to cattle on dry matter intake (DMI), milk production, milk composition, and efficiency of high-producing cows fed diets formulated to maximize milk fat.

Methods: Ninety-six lactating Holstein cows (36 primiparous, 60 multiparous; 106±17 days in milk, “DIM”) were balanced by parity, DIM, and milk production and were randomly assigned to 1 of 12 pens with 8 cows per pen. All cows received 11 g/ton Rumensin for a 5-week covariate period after which pens received 1 of 4 dietary treatments (n=3) formulated to provide 0 g/ton (Control, “CON”), 11 g/ton (R11), 14.5 g/ton (R14.5), or 18 g/ton (R18) of Rumensin for 9 weeks. Basal diet was 54% forage and 27% Neutral Detergent Fiber (NDF), 29% starch, and 2.3% Rumen Unsaturated Fatty Acid Load (RUFAL). The pen was the experimental unit, and data were analyzed using the Fit Model Procedure of JMP with treatment as a fixed effect and pen as a random effect. LS-means were determined and contrasts of CON vs. R11, CON vs. R14.5, CON vs. R18, and linear and quadratic effects were tested.

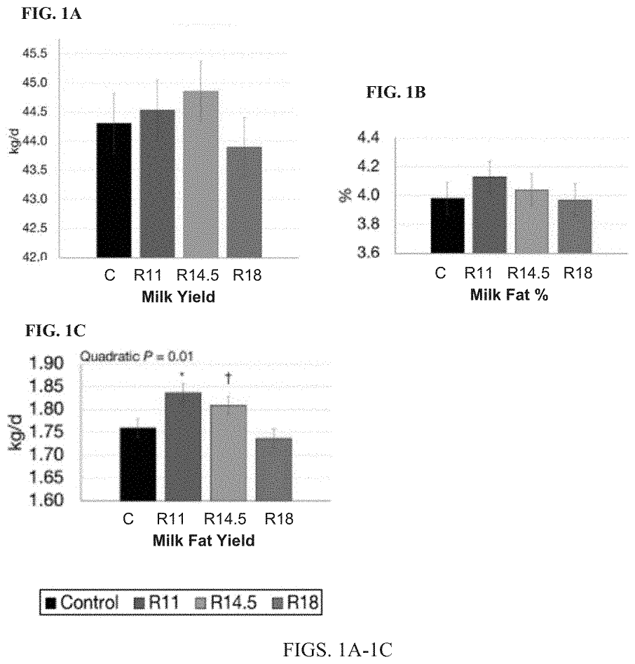

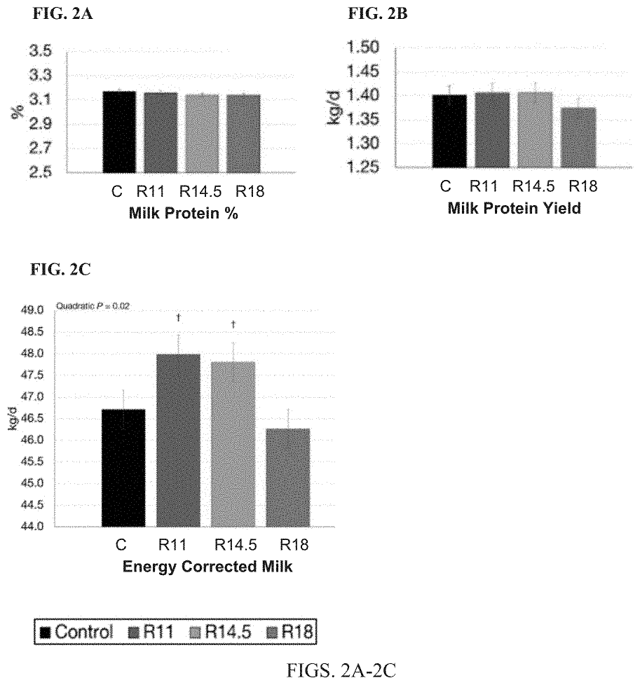

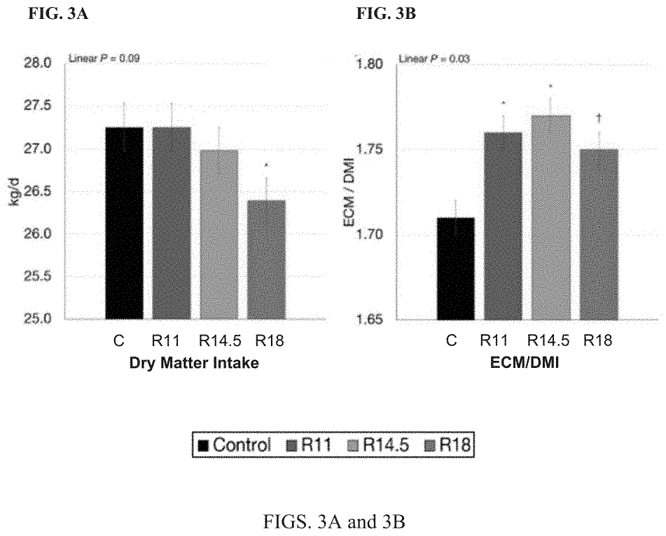

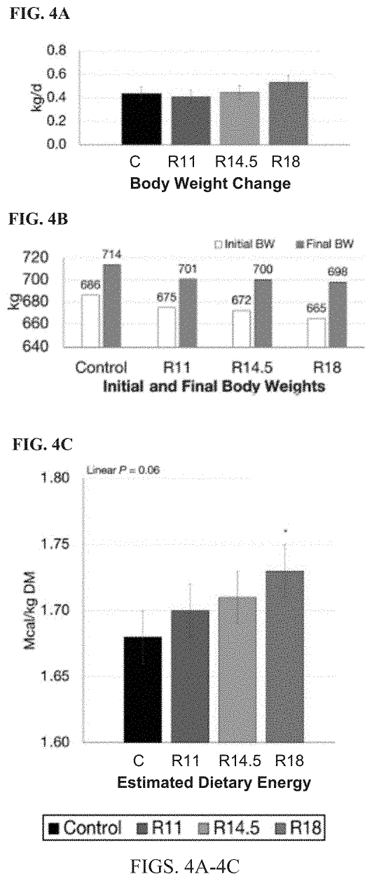

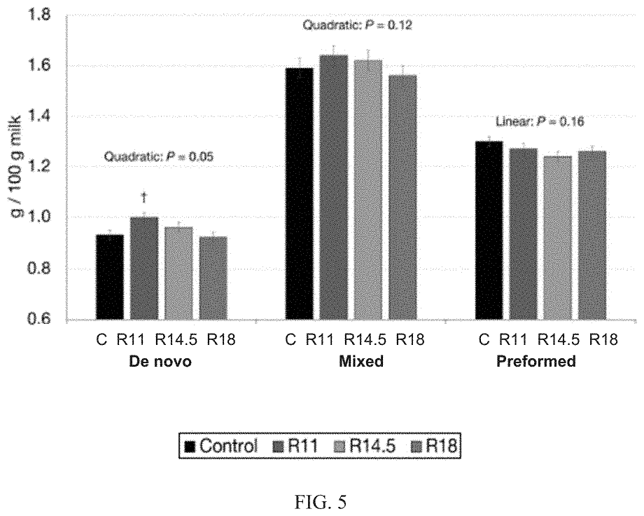

Results: Milk yield (44 kg/d), fat percentage (4.0%), protein percentage (3.2%), and yield (1.4 kg/d) were not affected by treatment (P 0.36), as illustrated by FIG. 1A, FIG. 1B, FIG. 2A, FIG. 2B, respectively. Milk fat yield was quadratic (P=0.01) and R11 increased yield 0.08 kg/d vs. CON (P=0.04), as illustrated by FIG. 1C. Rumensin quadratically affected energy corrected milk (ECM; P=0.02) and R11 increases ECM 1.3 kg/d vs CON (P=0.09), as illustrated by FIG. 2C. De novo fatty acid yield was quadratic (P=0.05) with R11 numerically highest vs. CON (0.99 vs 0.93 g/100 g milk; P=0.15), as illustrated by FIG. 5. DMI decreased linearly with increasing Rumensin level (P=0.09) and R18 cows ate less than CON (26.4 vs. 27.3 kg/d; P=0.05), as illustrated by FIG. 3A. Rumensin linearly increased ECM/DMI (P=0.03) with R11 and R14.5 cows increasing 2.9% (P=0.05) and 3.5% (P=0.02), respectively, and R18 increased 2.3% (P=0.09) vs. CON, as illustrated by FIG. 3B. Estimated dietary energy increased linearly with increasing Rumensin level (P=0.06) and was increased 3.0% in R18 vs. CON (P=0.01), as illustrated by FIG. 4C.

Conclusion: Administration of monensin improves ECM/DMI, estimated dietary energy, and does not negatively impact milk fat percentage. In particular, a monensin concentration of 14.5 g/ton (R14.5) shows improved milk yield compared to control, a monensin concentration of 11 g/ton (R11), and a monensin concentration of 18 g/ton (R18). R14.5 showed a lower fat percentage and fat yield than R11, while maintaining milk protein percentage and protein yield at roughly the same as the control and R11. R18 showed lower milk fat percentage, fat yield, and protein yield than the control, R11, and R14.5. R14.5 showed improved ECM/DMI over the control, R11, and R18. R14.5 also showed a slight body weight gain over R11 but a lower weight gain than R18, as illustrated by FIG. 4A and FIG. 4B. Estimated dietary energy for R14.5 fell between R11 and R18, as illustrated by FIG. 4C. R14.5 resulted in less de novo and mixed fatty acids than R11 and more de novo and fatty acids than R18. R14.5 resulted in less preformed fatty acids than the control, R11, and R18, as illustrated by FIG. 5.

### Example 2

### New York/Cornell Study

The instant example, referred to herein as the New York Study, provides evaluation of increasing dietary monensin (RUMENSIN®, Elanco Animal Health, Greenfield, Ind.) concentrations on milk and component yield, milk FA profile, and dry matter intake (DMI) in dairy cows fed contemporary diets. Lactating dairy cows were used for the instant example.

Methods: One-hundred ninety-two lactating cows (120±50 DIM) were randomly assigned to 12 pens each including 16 cows. Pens were assigned a treatment diet following a 4 week covariate period where all cows received 11 g/ton (dry matter basis, “DM” basis) monensin. The treatment diets included on a DM basis: 0 g/ton (CON), 11 g/ton (R11), 14.5 g/ton (R14.5), and 18 g/ton (R18) monensin. Diets included 35% corn silage, 19% grass haylage, 18% corn meal, 7% soybean meal, and 21% pre-mix containing monensin.

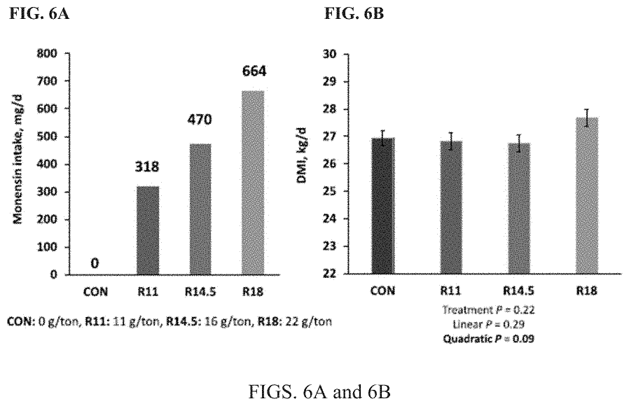

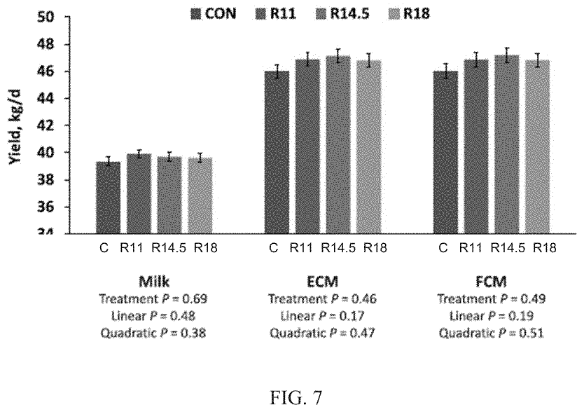

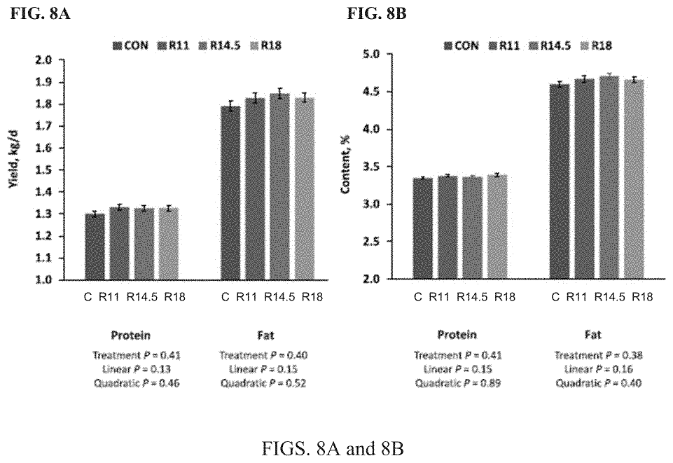

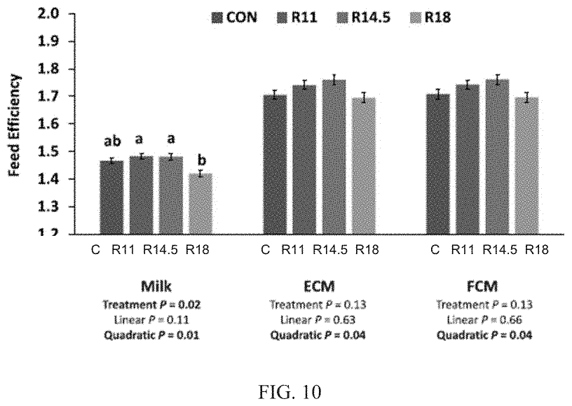

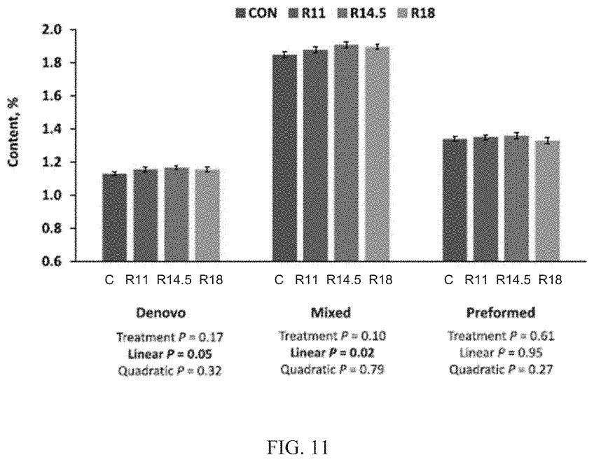

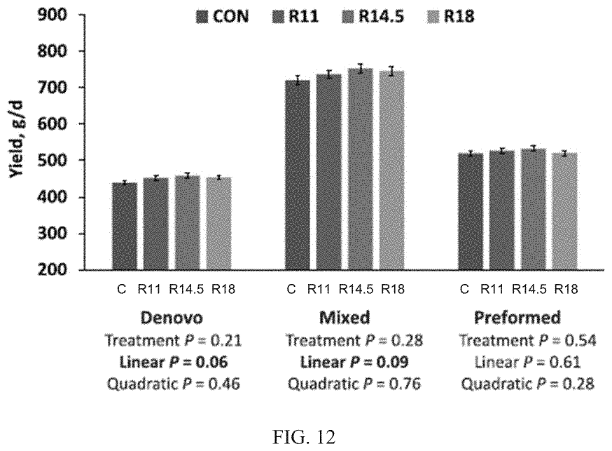

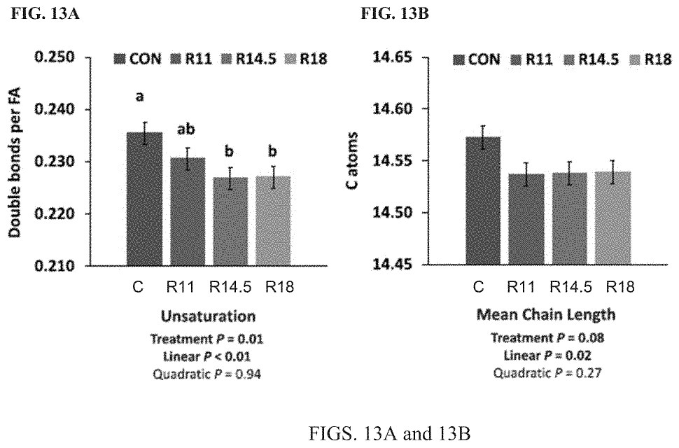

Results: The DMI increased numerically in the R18 group, as illustrated by FIG. 6. Milk yield was not affected by monensin treatment (39.3 kg/d for CON, 39.9 kg/d for the R11 group, 39.7 kg/d for the R14.5 group, and 39.6 kg/d for the R18 group), as illustrated by FIG. 7. Milk fat percentage was greater than 4.6% and milk true protein percent was equal to or greater than 3.35% for all treatment groups, as illustrated by FIG. 8B. Energy-corrected milk (ECM) was numerically greater in the R11 group, the R14.5 group, and the R18 group compared to CON (46.9, 47.1, 46.8 vs. 46.0 kg/d), as illustrated by FIG. 7. Milk fat yield (1.79 kg/d for CON, 1.83 kg/d for the R11 group, 1.85 kg/d for the R14.5 group, and 1.83 kg/d for the R18 group) and milk protein yield (1.30 kg/d for CON, 1.33 kg/d for the R11 group, 1.33 kg/d for the R14.5 group, and 1.33 kg/d for the R18 group) were not significantly different among treatments, as illustrated by FIG. 8A. There was a quadratic effect of monensin on milk production efficiency, ECM, and fat corrected milk (FCM), with the highest efficiency in the R11 and R14.5 groups, as illustrated by FIG. 10. In addition, milk de novo and mixed-origin FA yield were positively linearly affected by monensin feeding, while FA unsaturation was significantly reduced in the R14.5 group and the R18 group compared to CON, as illustrated in FIG. 13. Increasing monensin supplementation resulted in a non-significant 2% increase in ECM while linearly increasing FA yield, as illustrated by FIG. 12.

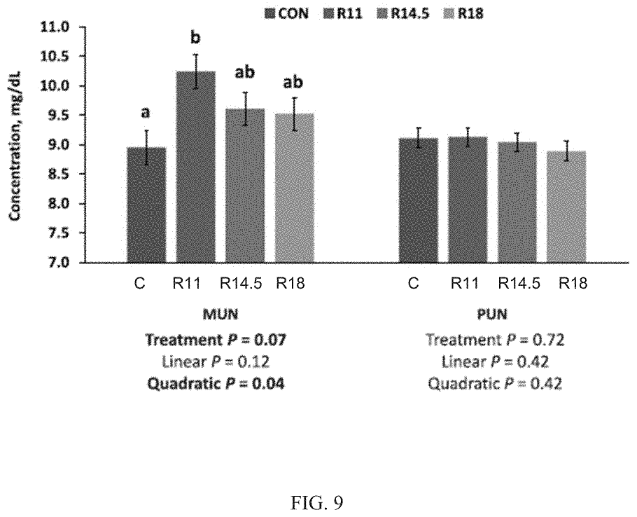

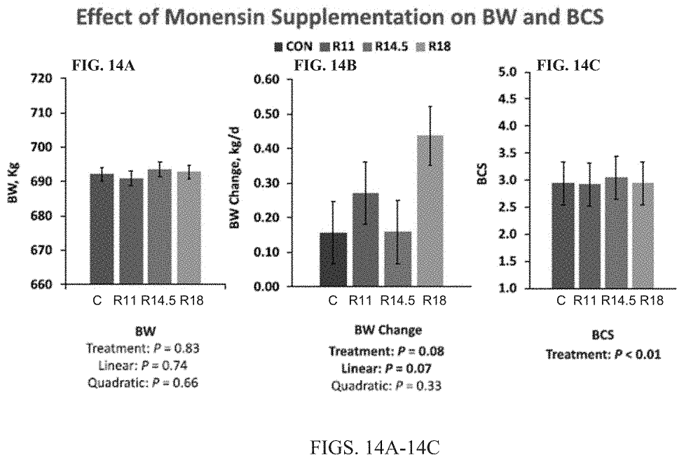

Conclusion: The results above show that, while monensin has little effect on milk yield, monensin causes a numerical increase in milk fat yield and content, and a numerical increase in ECM and FCM. Monensin at R11 and R14.5 showed a minor effect on DMI, with a slight increase in DMI for R18. Monensin numerically increases protein yield and content but has a tendency to increase MUN, as illustrated by FIG. 9. Monensin shows a quadratic effect on ECM feed efficiency (FE) and FCM FE, with a maximum at R14.5. However, a significant reduction in milk FE occurs at R18 as compared to R11 and R14.5. A tendency for a greater body weight (BW) change occurs in treatment weeks 0 to 5, as illustrated by FIG. 14. A linear increase in de novo and mixed fatty acid (FA) content and yield results from monensin supplementation, with no effect on preformed fatty acids, as illustrated by FIG. 12. Monensin also linearly reduces FA unsaturation and mean chain length, as illustrated by FIG. 13. In sum, the data show that monensin can be fed at higher concentrations to achieve high milk component yield in lactating dairy cows fed contemporary diets, with R14.5 showing a maximum quadratic effect on ECM FE and FCM FE.

### Example 3

### Comparative Analysis of Wisconsin and New York Studies

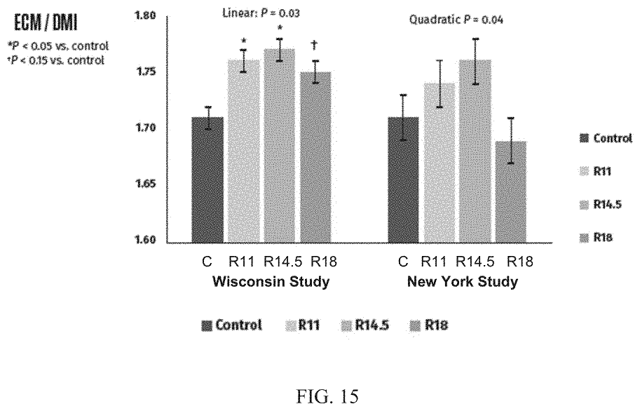

The instant example provides analysis of the two studies (the Wisconsin Study and New York Study) and demonstrates that particular monensin levels R11 (about 11 g/ton) and R14.5 (about 14.5 g/ton) quadratically increased milk production efficiency in mid-lactation cows, as illustrated by FIG. 15. Both R11 and R14.5 showed increased milk production efficiency (ECM/DMI) over both the control (0 g/ton) and higher levels of monensin (R18, about 18 g/ton) within the range.

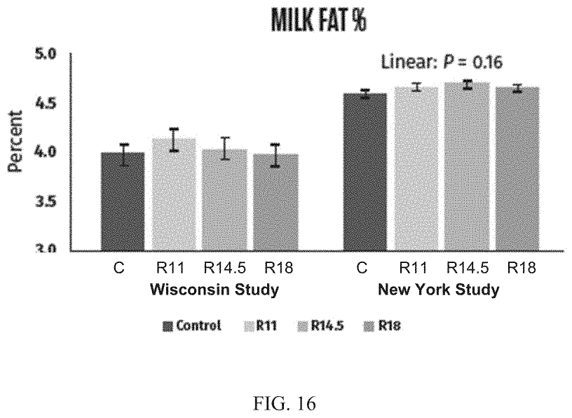

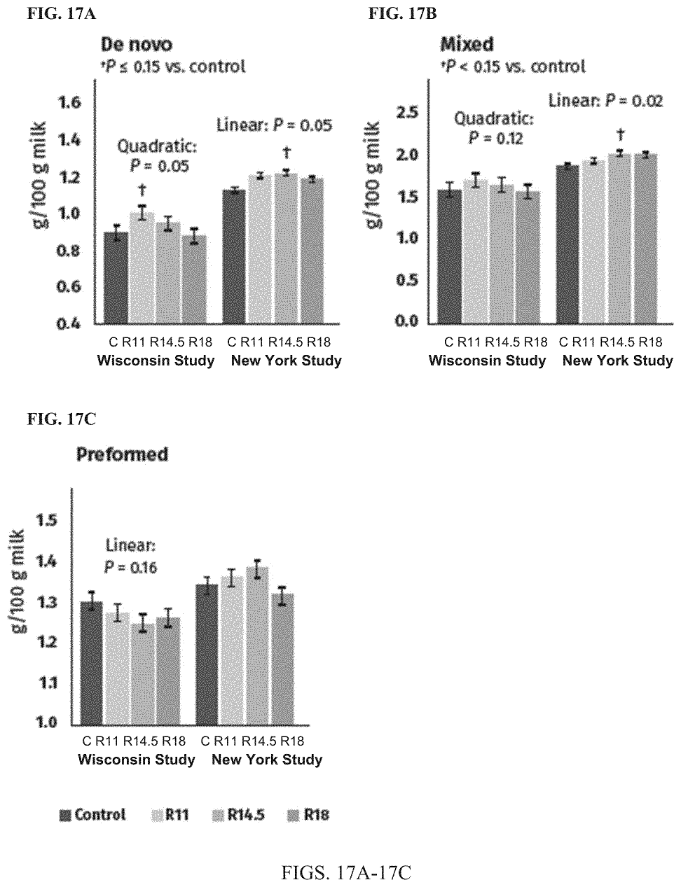

Moreover, the same studies show that milk fat content was not negatively affected, but de novo and mixed fatty acid yields were increased, as illustrated by FIG. 16 and FIG. 17. For instance, as illustrated by FIG. 16, both studies show a statistically insignificant increase in milk fat percentage over the control for both R11 and R14.5.

As illustrated by FIG. 17, the Wisconsin Study showed quadratic increases in de novo fatty acids for both R11 and R14.5 over the control and R18. Specifically, the Wisconsin Study showed the highest increase in de novo fatty acids for R11, from 0.93 g/100 g milk for the control to 0.99 g/100 g milk for R11. The New York Study showed linear increases in de novo fatty acids for R11 and R14.5, with R14.5 showing a slightly higher increase than R11 over the control, and with R18 showing a decrease over the control.

### Example 4

### Combined Analysis of Wisconsin and New York Studies

The instant example provides the combined analysis of data from the two studies (Wisconsin Study and New York Study). The two studies were combined statistically to explore effects on milk production efficiency and other variables associated with efficiency. By effectively doubling pen numbers an increase in power of the analysis was provided.

Combination of experimental datasets evaluated monensin concentrations with respect to improvements in milk components and milk production efficiency. All treatments, including the control diet, produced above average milk protein and fat over industry standards. As described below, the 14.5 g/ton treatment exhibited the highest level of component corrected milk yield and feed efficiency.

Summary: The combined statistical analysis of the two studies (Wisconsin Study and New York Study) demonstrated a linear improvement in milk fat production when monensin is applied to the diet, particularly improving de novo and mixed fatty acid production per unit of milk. Milk yield did not change among treatments; however, the proportion of milk fat produced, improved milk fat production in monensin fed cattle over the control diet. Subsequently, milk fat yield, component corrected milk yield, and feed efficiency improved for diets fed the 11 and 14.5 g/ton monensin diets. Further, quadratic effects for feed efficiency were found, with the 14.5 g/ton diet having the highest level of feed efficiency over all other treatments.

Analysis of these combined datasets was performed in various ways, with the inclusion of either weekly milk yield averages or sample day milk yields to calculate milk component yields, component corrected yields, and feed efficiency. Further, the combined dataset was analyzed using data collected from all experimental weeks as well as the exclusion of data from the first three weeks of experimental treatment feeding. This was done to exclude any shifts in response variables while the rumen environment was adapting to the treatment provided.

Materials & Methods: All data from both experimental locations were combined and statistically analyzed as a longitudinal experiment design through SAS version 9.4 (SAS Institute Inc., Cary, N.C.). Experimental response variables, excluding body condition score (BCS), were analyzed using PROC MIXED and LSMEAN statements to compare treatment means using the following model:

γᵢⱼₖₗₘ=μ+Tᵢ+Wⱼ+TWᵢⱼ+Lₖ+P_{l(i*k)}+WPⱼₗ(i*k)+C_{m(l*i*k)}BXₘₗᵢₖ+∈₍ᵢⱼₖₗ₎ₘ

Where γᵢⱼₖₗₘ=an individual observation of the response variable analyzed, Tᵢ=the i^{th} dietary treatment, Wⱼ=the j^{th} experimental week, TWᵢⱼ=the interaction of the i^{th} treatment with the j^{th} experimental week, Lₖ=the k^{th} experimental location, P_{l(i*k)}=the 1^{th} pen within the i^{th} treatment and k^{th} experimental location, WP_{jl(i*k)}=the interaction of the j^{th} experimental week with the 1^{th} pen within the i^{th} treatment and k^{th} experimental location, C_{m(l*i*k)}=the m^{th} cow within the 1^{th} pen, the i^{th} treatment and k^{th} experimental location, BXₘₗᵢₖ=the covariate observation obtained after three weeks of feeding a common diet to all treatment groups, and ∈ᵢⱼₖₗₘₙ=the random error of the m^{th} cow within the 1^{th} pen, the i^{th} treatment and the k^{th} location. Under this model, all response data was examined for outliers, where any observation which produced a studentized residual greater than 3 or less than −3 was excluded from analysis and the response variable was then reran for statistical interpretation. Data were analyzed with pen as the experimental unit and cow as the observational unit. Dietary treatment and experimental week were considered fixed effects, whereas location, pen and cow were considered random effects. An auto-regressive covariance structure [AR(1)] was used to analyze repeated measurements (week of experimental period) with cow nested in pen within treatment as the subject. For pen level observations (DMI, production efficiency and NE_{L} measurements), the main effect of cattle was dropped from the statistical model above. Further, pen was used as the subject of the auto-regressive repeated analysis. Degrees of freedom were adjusted using the Kenward-Roger option and multiple treatment LSMEAN comparisons were adjusted by the Tukey method. Non-parametric analysis of BCS measurements, via PROC NPAR1WAY, was performed to evaluate differences in dietary treatments at both the initiation of the experiment and during the final week of the experiments. Statistical significance was reported as P≤0.05 and tendencies as 0.05<P≤0.10.

Given the mode of action monensin plays on the rumen environment and the way in which both experiments were designed, a follow-up on the statistical analysis was performed to evaluate dietary treatment effects when the rumen environment has adapted to the concentration of monensin in the diet, assumed to be 21 days post feeding initiation. This analysis, deemed the ‘post-acclimation’ analysis, removed the first three experimental weeks of data from the experimental dataset, leaving only the fourth to ninth weeks for statistical interpretation. The statistical model was identical to the previously described approach and all data, except for body weight and NE_{L} (due to missing data), were reanalyzed for interpretation.

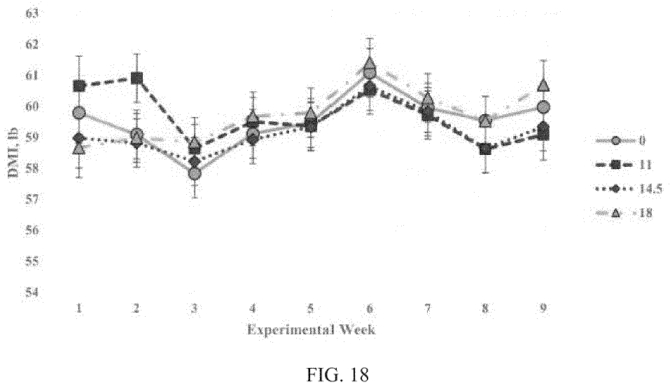

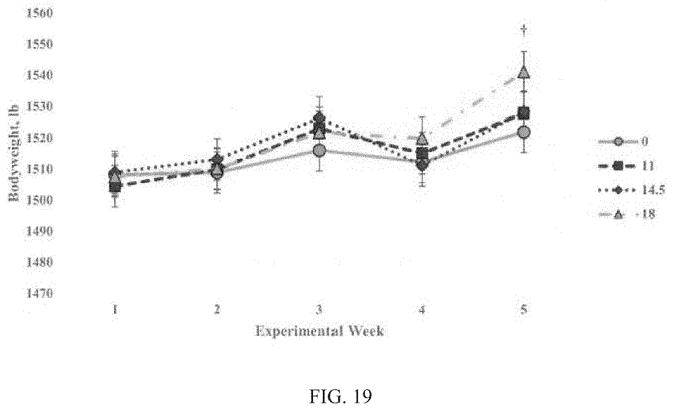

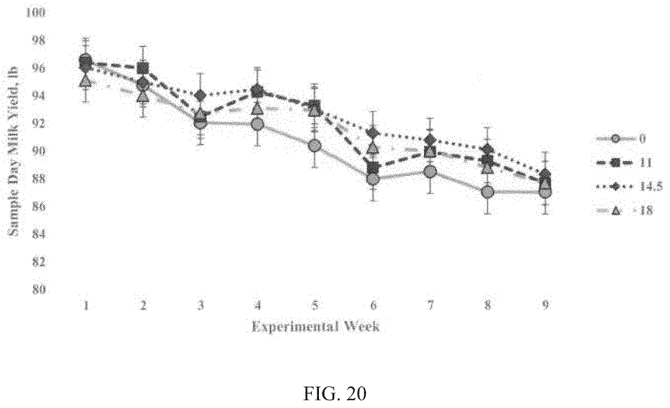

Results: Dry matter intake was not different among dietary treatments, either during the entire experiment (P=0.87; Table 2) or during the post-acclimation period (P=0.74; Table 5) but did change over time (P<0.01; FIG. 18). This lack of difference is consistent with previous research involving monensin feeding. Similarly, bodyweight and body weight change were not different among dietary treatments yet did change throughout the experiment (P<0.01; FIG. 19 and FIG. 20). The 18 g/ton treatment had a numerically higher rate of bodyweight gain and is believed to be an artifact of a higher rate of gain observed in the New York Study animals which was not believed to be influenced by the treatments administered.

Milk yield data were analyzed two individual ways, including weekly average yield (Table 3) and yield produced on the day of milk sampling (Table 4). Although weekly average milk yield was not significantly different among dietary treatments (P=0.53), the yield produced on the day of sampling showed a linear tendency (P=0.06) with a greater inclusion of monensin improving yield. This tendency was improved to a statistical significance (P=0.03) when the post-acclimation data was analyzed, with a range of 1.5 pounds of milk difference from the control treatment to the 18 g/ton treatment (Table 7). This improvement in yield is consistent with other studies.

Temporal data of sample day milk yield is shown in FIG. 20 and indicates significant differences across the experiment for all treatments (P<0.01) with no significant treatment by time interaction (P=0.75). Average weekly milk yield did begin to approach a statistical tendency for treatment by time interaction (P=0.18); however, this is likely due to the changes in yield throughout the first three weeks of the experiment, as post-acclimation statistical analysis did not show statistical significance for either the weekly average milk yield (P=0.95; Table 6) and day of sampling yield (P=0.92; Table 7). It is worth noting that from experimental week 5 to 6 there was an observable decrease in yield for the 11 g/ton treatment and has subsequent effect on all other component yield, corrected yield, and efficiency data (FIG. 20).

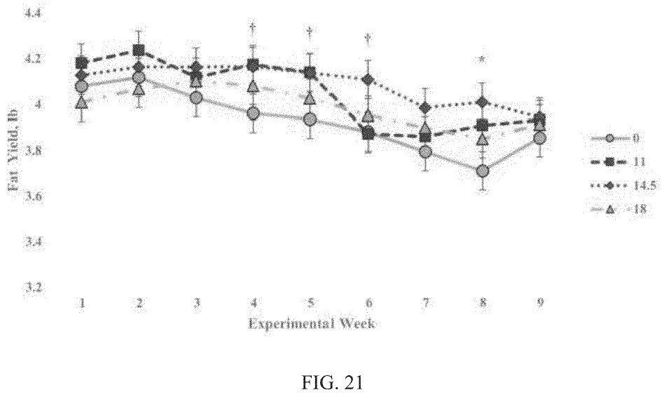

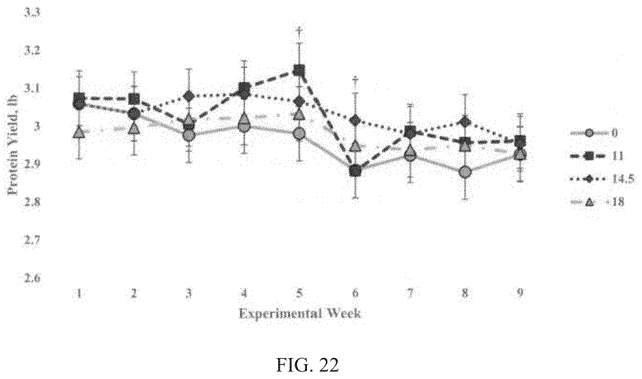

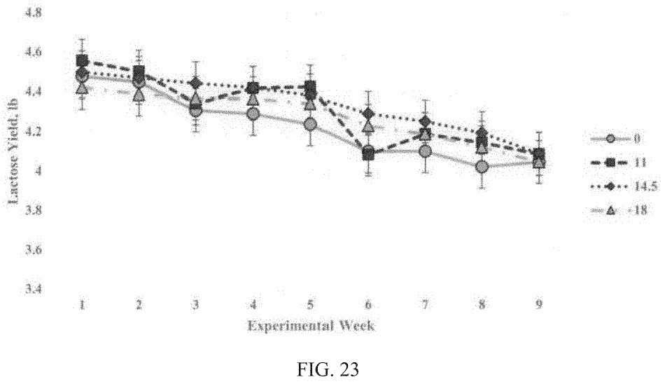

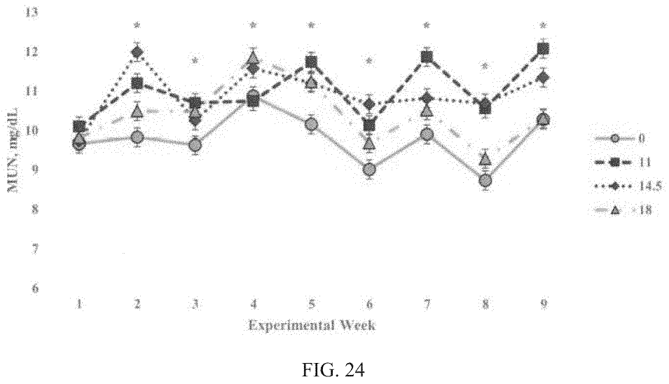

Milk protein percentage was not different among treatments either throughout the entirety of the study (P=0.83; Table 2) or through the post-acclimation analysis (P=0.82; Table 5), with all treatments demonstrating a high level of protein (3.29% over the entre study and 3.32% throughout the post-acclimation period). Milk protein yield exhibited a linear tendency (P=0.09) when using sample day milk yield and analyzing the post-acclimation dataset (Table 7 & FIG. 22)), demonstrating an increase in protein yield with increasing monensin concentration. All other analysis of milk protein were not affected by the level of monensin in the diet, following a similar trend as what has been previously observed in monensin feeding studies. Alternatively, milk fat percent demonstrated a linear significance and tendency for monensin treatment over the entire experiment (P=0.05; Table 2) and the post-acclimation period (P=0.06; Table 5), respectively. Further, monensin treatment tended to be significant for milk fat yield when applying weekly average milk (P=0.07; Table 3) and sample day yield (P=0.07; Table 4 & FIG. 21) over the entire period and was statistically significant for the post-acclimation dataset (P=0.05; Table 6 & P=0.04; Table 7). These results suggest that greater inclusion of monensin in the diet improves milk fat output, which contradicts outcomes of previous research demonstrating a reduction in milk fat production. De novo and mixed fatty acid production, expressed as g/100 g of milk, was linearly significant over the entire experiment (P=0.01 and P=0.05 for de novo and mixed, respectively; Table 2) and throughout the post-acclimation period (P=0.01 and P=0.02 for de novo and mixed, respectively; Table 5). This linear trend also suggests an improvement in both classes of fatty acids with increasing monensin concentration. Conversely, preformed fatty acid production exhibited a linear tendency (P=0.09; Table 2) over throughout the experiment, with increasing monensin concentrations lowering preformed fatty acids. No difference were observed for preformed fatty acids during the post-acclimation period (Table 5) and for total milk fatty acid production (Table 2 and Table 5). In contradiction to this report, other studies have reported significant decreases in de novo FA concentration per total FA with monensin treatment, and other studies demonstrated no differences in mixed or preformed fatty acid production relative to total fatty acids. Milk urea nitrogen was different among treatments during throughout the entire experiment (P<0.01; Table 2 and FIG. 24) and the post-acclimation period (P<0.01; Table 5). Further, a quadratic tendency (P=0.07; Table 2) was shown, indicating that the 11 g/ton treatment tended to have a higher milk urea nitrogen concentration over all other treatments. Other studies have found monensin had no effect on milk urea nitrogen while some studies have reported an increase in milk urea nitrogen with monensin treatment. An alternative meta-analysis has reported blood, plasma, and serum concentration increased with monensin treatment.

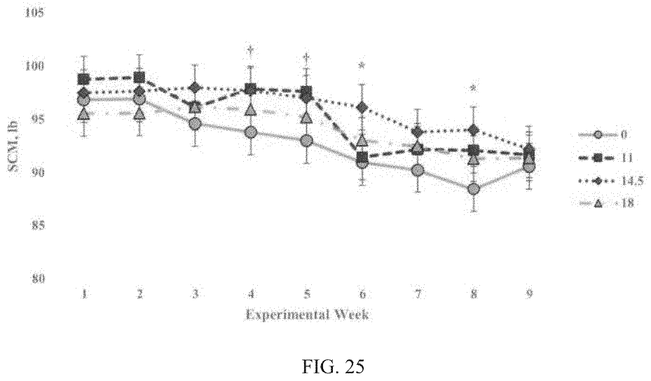

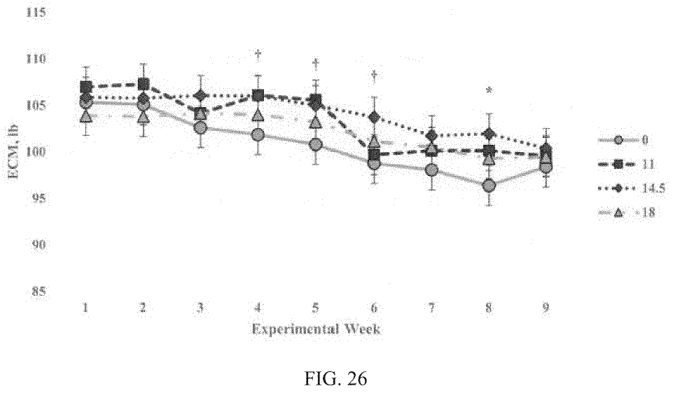

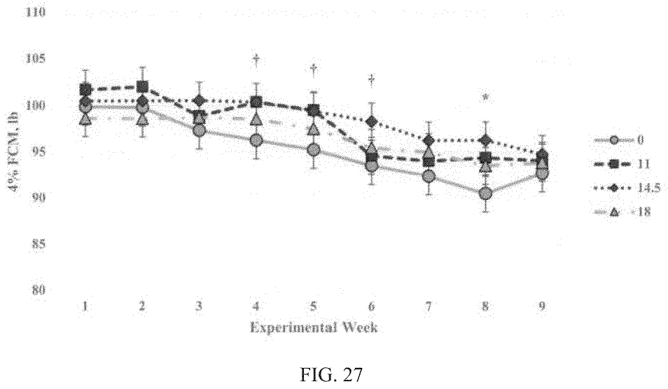

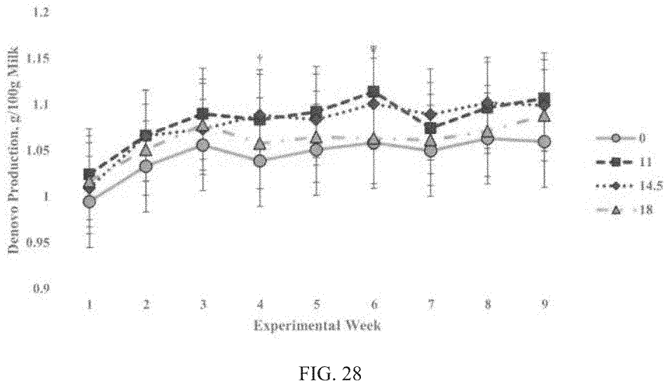

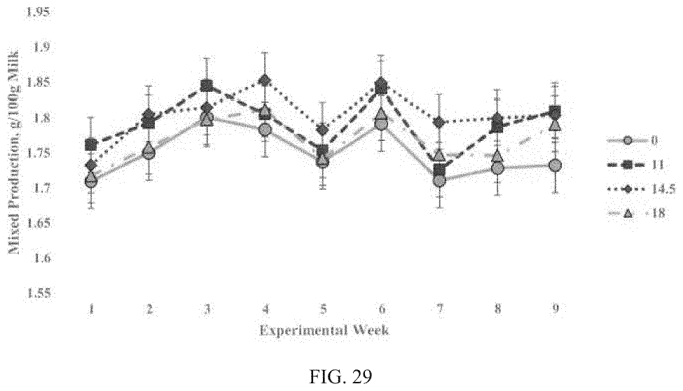

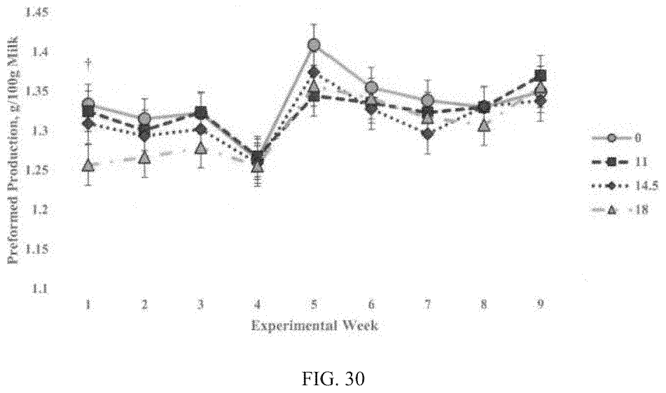

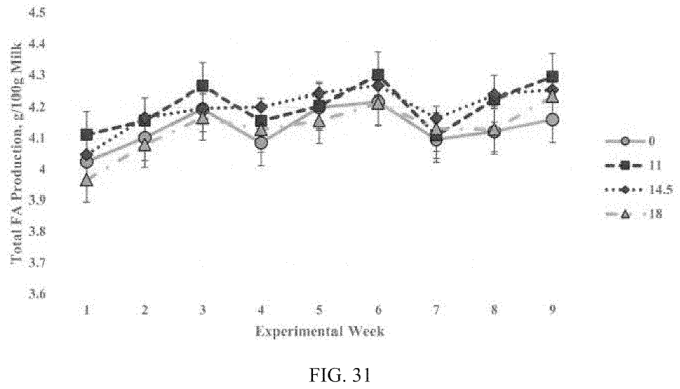

Solids corrected milk (SCM), energy corrected milk (ECM) and 4% fat corrected milk (FCM) all exhibited similar statistical trends (Table 3, Table 4, Table 6 and Table 7). Using weekly averaged milk yield to calculate these parameters produced a linear tendency throughout the experiment for SCM (P=0.07) and FCM (P=0.09) and statistical differences during the post-acclimation period for SCM (P=0.02), ECM (P=0.03) and FCM (P=0.03). Using sample day milk yield followed a similar pattern, with linear significance for SCM, ECM and FCM (P=0.02; Table 4) throughout the experiment and improved significance throughout the post acclimation period (P=0.01; Table 7). In each of these analyses, a linear increase in corrected milk yields was found when adding a greater concentration of monensin was added to the diet; however, the highest level of yield was found at the 14 g/ton treatment. The post-acclimation period demonstrated that, throughout the remainder of the experiment, the 14.5 g/ton treatment demonstrated numerically greater yield of SCM (FIG. 25), ECM (FIG. 26) and FCM (FIG. 27) over all other treatments. Further, the 11 g/ton and 18 g/ton produced similar levels of corrected milk, with the control diet, without any monensin in the diet, having the lowest yield. Net energy of lactation (NE_{L}) was calculated according to the NRC 2001 equation, substituting weekly average milk yield (Table 3) and sample day milk yield (Table 4). Both analyses yield similar interpretation, with the 18 g/ton treatment having a numerically higher NE_{L} over all other treatments, yet no statistical differences were detected (P>0.10). A numerically higher NE_{L} for the 18 g/ton diet is attributed to an increased bodyweight gain observed in the cattle in the New York Study, as this study's report described abnormally high rates of bodyweight gain and was believed to not be attributed to monensin treatment.

Feed efficiency was calculated using fluid milk yield and component corrected milk yield over DMI. Similar to corrected milk yield results, feed efficiency was highest for the 14.5 g/ton throughout the experiment and post-acclimation period, regardless of whether weekly average milk yield or sample day milk yield was used for calculation. Contrary to this analysis, other studies have found production efficiencies to be significantly greater in cows fed 18 g/ton monensin compared to no monensin. When ranking all measurements of feed efficiency, the 11 g/ton treatment slightly lower feed efficiency over the 14.5 g/ton treatment, with the control diet and 18 g/ton treatment exhibiting the lowest, yet similar, efficiencies. The lower efficiency in the 18 g/ton treatment is likely attributed to the numerically higher DMI observed with numerically lower milk yield over all other diets receiving monensin.

Conclusions: Dietary inclusion of monensin improved milk component yield over cattle consuming the control diet. De novo and mixed milk fatty acid production was linearly increased with monensin concentration whereas preformed fatty acid production tended to linearly decrease with monensin concentration. Solids corrected milk, ECM, and FCM linearly increased with monensin concentration; however, the interpretation of which treatment produced the numerically largest component corrected yield was based on whether the entire dataset or post-acclimation dataset was analyzed. Feed efficiency exhibited a quadratic effect and was numerically highest in cattle fed the 14.5 g/ton monensin treatment, suggesting an optimum dosage exists in regard to efficient production.

TABLE 1
Milk production efficiency data for both studies.

| | Wisconsin Study | | | | New York Study | | | |
|---|---|---|---|---|---|---|---|---|
| | Control | R11 | R14.5 | R18 | Control | R11 | R14.5 | R18 |
| Milk Yield (lb/d) | 98 | 98 | 99 | 97 | 86 | 88 | 87 | 87 |
| ECM (lb/d) | 103 | 106 | 105 | 102 | 101 | 103 | 104 | 103 |
| Milk Fat (%) | 3.98 | 4.13 | 4.04 | 3.97 | 4.60 | 4.67 | 4.71 | 4.67 |
| Protein (%) | 3.17 | 3.16 | 3.14 | 3.14 | 3.35 | 3.38 | 3.37 | 3.39 |
| DMI (lb/d) | 60 | 60 | 60 | 58 | 59 | 59 | 59 | 61 |
| Weight Gain (lb/d) | 0.96 | 0.90 | 0.98 | 1.17 | 0.35 | 0.60 | 0.35 | 0.97 |
| Estimated Dietary Energy (Mcal/kg DM) | 1.68 | 1.70 | 1.71 | 1.73 | 1.64 | 1.65 | 1.65 | 1.68 |

TABLE 2
Dry matter intake, body weight, body condition score, milk yield and component proportions and milk fatty acid
results throughout entire experiment for lactating dairy cattle fed varying concentrations of monensin.

| | Sample | Dietary Treatment | | | | | Contrasts | | P-Values |
|---|---|---|---|---|---|---|---|---|---|
| Parameter | Size | 0 | 11 | 14.5 | 18 | SEM | Linear | Quad | Covariate |
| DMI | 51 | 59.5 | 59.7 | 59.2 | 59.7 | 0.52 | 0.82 | 0.51 | <0.01 |
| BW | 330 | 1513.6 | 1516.3 | 1517.7 | 1520.3 | 5.1 | 0.29 | 0.83 | <0.01 |
| BW Delta | 117 | 1.14 | 1.05 | 1.02 | 1.33 | 0.46 | 0.99 | 0.94 | 0.88 |
| Initial BCS | 72 | 3.21 | 3.04 | 3.22 | 3.20 | — | — | — | — |
| Final BCS | 72 | 2.89 | 2.93 | 2.96 | 2.87 | — | — | — | — |
| Milk Production Results | | | | | | | | | |
| Protein, % | 620 | 3.28 | 3.29 | 3.30 | 3.29 | 0.02 | 0.42 | 0.95 | <0.01 |
| Fat, % | 618 | 4.36 | 4.45 | 4.47 | 4.42 | 0.04 | 0.05 | 0.71 | <0.01 |
| Lactose, % | 617 | 4.65 | 4.66 | 4.67 | 4.66 | 0.03 | 0.16 | 0.98 | <0.01 |
| MUN, mg/dL | 623 | 9.8^{ab} | 11.0^{b} | 10.9^{b} | 10.4^{ab} | 0.19 | <0.01 | 0.07 | <0.01 |
| Milk Fatty Acid Results | | | | | | | | | |
| De novo, g/100 g | 623 | 1.04^{a} | 1.08^{b} | 1.08^{b} | 1.06^{ab} | 0.05 | 0.01 | 0.18 | <0.01 |
| Mixed, g/100 g | 622 | 1.75 | 1.79 | 1.80 | 1.77 | 0.03 | 0.05 | 0.86 | <0.01 |
| Performed, g/100 g | 621 | 1.33 | 1.32 | 1.31 | 1.30 | 0.01 | 0.09 | 0.86 | <0.01 |
| Total FA, g/100 g | 620 | 4.13 | 4.20 | 4.20 | 4.13 | 0.06 | 0.31 | 0.66 | <0.01 |
| De novo, Relative | 626 | 25.22^{a} | 25.69^{b} | 25.61^{b} | 25.48^{ab} | 0.81 | 0.01 | 0.10 | <0.01 |
| Mixed, Relative | 624 | 42.65^{ab} | 42.65^{ab} | 42.85^{b} | 42.60^{ab} | 0.22 | 0.01 | 0.95 | <0.01 |
| Preformed, Relative | 625 | 32.54^{b} | 31.95^{ab} | 31.52^{a} | 31.95^{ab} | 0.99 | <0.01 | 0.50 | <0.01 |

| | P-Values | | | Random Effect | | | | |
|---|---|---|---|---|---|---|---|---|
| Parameter | Treatment | Week | TxW | Location | Pen(LxT) | PenxW(LxT) | AR(I) | Residual |
| DMI | 0.87 | <0.01 | 0.95 | 0.46 | NIA | NIA | <0.01 | <0.01 |
| BW | 0.65 | <0.01 | 0.81 | 0.30 | — | 0.02 | <0.01 | <0.01 |
| BW Delta | 0.96 | <0.01 | 0.83 | 0.50 | — | 0.11 | <0.01 | <0.01 |
| Initial BCS | <0.01 | — | — | — | — | — | — | — |
| Final BCS | 0.55 | — | — | — | — | — | — | — |
| Milk Production Results | | | | | | | | |
| Protein, % | 0.83 | <0.01 | 0.28 | — | 0.06 | <0.01 | <0.01 | <0.01 |
| Fat, % | 0.13 | <0.01 | 0.93 | 0.32 | 0.23 | 0.04 | <0.01 | <0.01 |
| Lactose, % | 0.54 | <0.01 | 0.49 | 0.25 | 0.21 | <0.01 | <0.01 | <0.01 |
| MUN, mg/dL | <0.01 | <0.01 | <0.01 | | <0.01 | <0.01 | <0.01 | <0.01 |
| Milk Fatty Acid Results | | | | | | | | |
| De novo, g/100 g | 0.01 | <0.01 | 1.00 | 0.24 | 0.38 | <0.01 | <0.01 | <0.01 |
| Mixed, g/100 g | 0.11 | <0.01 | 0.99 | 0.26 | 0.16 | <0.01 | <0.01 | <0.01 |
| Performed, g/100 g | 0.26 | <0.01 | 0.98 | 0.31 | 0.48 | <0.01 | <0.01 | <0.01 |
| Total FA, g/100 g | 0.23 | <0.01 | 0.89 | 0.26 | 0.19 | 0.06 | <0.01 | <0.01 |
| De novo, Relative | 0.01 | <0.01 | 0.18 | 0.24 | 0.14 | <0.01 | <0.01 | <0.01 |
| Mixed, Relative | 0.06 | <0.01 | 0.06 | 0.28 | 0.05 | <0.01 | <0.01 | <0.01 |
| Preformed, Relative | 0.01 | <0.01 | 1.00 | 0.24 | 0.35 | <0.01 | <0.01 | <0.01 |

^{ab}Within a row, means without a common superscript differ (P < 0.05).

TABLE 3
Weekly averaged milk and component yield, corrected milk yield, feed efficiency
and net energy of lactation changes throughout entire experiment for
lactating dairy cattle fed varying concentrations of monensin.

| | Sample | Treatment | | | | | Contrasts | | P-Values |
|---|---|---|---|---|---|---|---|---|---|
| Parameter | Size | 0 | 11 | 14.5 | 18 | SEM | Linear | Quad | Covariate |
| Milk Yield, lb | 630 | 90.4 | 91.5 | 91.0 | 90.4 | 0.76 | 0.64 | 0.44 | <0.01 |
| Protein, lb | 619 | 2.94 | 3.00 | 2.98 | 2.95 | 0.02 | 0.46 | 0.42 | <0.01 |
| Fat, lb | 619 | 3.91 | 4.03 | 4.03 | 3.95 | 0.04 | 0.07 | 0.52 | <0.01 |
| Lactose, lb | 620 | 4.20 | 4.27 | 4.26 | 4.22 | 0.03 | 0.33 | 0.52 | <0.01 |
| SCM, lb | 618 | 92.3^{x} | 94.7^{xy} | 94.5^{y} | 93.1^{x} | 0.69 | 0.07 | 0.38 | <0.01 |
| ECM, lb | 618 | 100.3 | 102.8 | 102.4 | 100.9 | 0.72 | 0.11 | 0.36 | <0.01 |
| FCM, lb | 620 | 94.8 | 97.2 | 96.9 | 95.5 | 0.70 | 0.09 | 0.37 | <0.01 |
| Milk:Feed | 50 | 1.56 | 1.57 | 1.57 | 1.53 | 0.01 | 0.82 | 0.73 | <0.01 |
| SCM:Feed | 51 | 1.56^{abx} | 1.59^{by} | 1.61^{b} | 1.56^{a} | 0.02 | 0.03 | 0.81 | <0.01 |
| ECM:Feed | 51 | 1.70^{ab} | 1.73^{aby} | 1.74^{b} | 1.69^{ax} | 0.02 | 0.06 | 0.86 | <0.01 |
| FCM:Feed | 51 | 1.60^{a} | 1.64^{b} | 1.65^{b} | 1.60^{a} | 0.01 | 0.02 | 0.94 | <0.01 |
| NE_{L} | 29 | 1.65 | 1.53 | 1.54 | 2.38 | 0.35 | 0.64 | 0.75 | 0.24 |

| | P-Values | | | Random Effect | | | | |
|---|---|---|---|---|---|---|---|---|
| Parameter | Treatment | Week | TxW | Location | Pen(LxT) | PenxW(LxT) | AR(I) | Residual |
| Milk Yield, lb | 0.53 | <0.01 | 0.18 | 0.33 | 0.32 | <0.01 | <0.01 | <0.01 |
| Protein, lb | 0.36 | <0.01 | 0.05 | — | 0.12 | <0.01 | <0.01 | <0.01 |
| Fat, lb | 0.07 | <0.01 | 0.85 | — | 0.15 | 0.08 | <0.01 | <0.01 |
| Lactose, lb | 0.41 | <0.01 | 0.34 | — | 0.41 | <0.01 | <0.01 | <0.01 |
| SCM, lb | 0.07 | <0.01 | 0.55 | — | 0.22 | 0.01 | <0.01 | <0.01 |
| ECM, lb | 0.08 | <0.01 | 0.47 | — | 0.18 | 0.02 | <0.01 | <0.01 |
| FCM, lb | 0.08 | <0.01 | 0.66 | — | 0.20 | 0.02 | <0.01 | <0.01 |
| Milk:Feed | 0.15 | <0.01 | 0.72 | — | N/A | N/A | <0.01 | <0.01 |
| SCM:Feed | <0.01 | <0.01 | 0.79 | 0.27 | N/A | N/A | <0.01 | <0.01 |
| ECM:Feed | <0.01 | <0.01 | 0.70 | 0.29 | N/A | N/A | <0.01 | <0.01 |
| FCM:Feed | <0.01 | <0.01 | 0.71 | 0.31 | N/A | N/A | <0.01 | <0.01 |
| NE_{L} | 0.23 | <0.01 | 0.72 | — | N/A | N/A | 0.08 | <0.01 |

^{ab}Within a row, means without a common superscript differ (P < 0.05).
^{xy}Within a row, means without a common superscript differ (0.05 ≤ P ≤ 0.10).

TABLE 4
Sample day milk and component yield, corrected milk yield, feed efficiency
and net energy of lactation changes throughout entire experiment for
lactating dairy cattle fed varying concentrations of monensin.

| | Sample | Treatment | | | | | Contrasts | | P-Values |
|---|---|---|---|---|---|---|---|---|---|
| Parameter | Size | 0 | 11 | 14.5 | 18 | SEM | Linear | Quad | Covariate |
| Day of Sample | 616 | 90.7 | 92.0 | 92.6 | 91.6 | 1.36 | 0.06 | 0.92 | <0.01 |
| Milk Yield, lb | | | | | | | | | |
| Protein, lb | 610 | 2.96 | 3.02 | 3.03 | 2.98 | 0.06 | 0.15 | 0.83 | <0.01 |
| Fat, lb | 610 | 3.93^{x} | 4.05^{xy} | 4.09^{y} | 3.99^{xy} | 0.06 | 0.03 | 0.90 | <0.01 |
| Lactose, lb | 611 | 4.22 | 4.30 | 4.34 | 4.27 | 0.10 | 0.06 | 0.93 | <0.01 |
| SCM, lb | 610 | 92.9^{x} | 95.2^{xy} | 96.0^{y} | 94.1^{xy} | 1.80 | 0.02 | 0.87 | <0.01 |
| ECM, lb | 610 | 100.8^{a} | 100.8^{a} | 104.1^{b} | 102.2^{ab} | 1.82 | 0.02 | 0.82 | <0.01 |
| FCM, lb | 610 | 95.3^{a} | 95.3^{a} | 98.6^{b} | 96.7^{ab} | 1.69 | 0.02 | 0.84 | <0.01 |
| Milk:Feed | 51 | 1.56^{xy} | 1.56^{xy} | 1.61^{y} | 1.55^{x} | 0.03 | 0.19 | 0.05 | <0.01 |
| SCM:Feed | 51 | 1.57^{a} | 1.59^{ab} | 1.64^{b} | .157^{a} | 0.04 | <0.01 | 0.04 | <0.01 |
| ECM:Feed | 51 | 1.70^{a} | 1.72^{a} | 1.77^{b} | 1.71^{a} | 0.04 | <0.01 | 0.05 | <0.01 |
| FCM:Feed | 51 | 1.60^{a} | 1.63^{a} | 1.68^{b} | 1.61^{a} | 0.04 | <0.01 | 0.03 | <0.01 |
| NE_{L} | 29 | 1.66 | 1.53 | 1.55 | 2.39 | 0.35 | 0.63 | 0.77 | 0.24 |

| | P-Values | | | Random Effect | | | | |
|---|---|---|---|---|---|---|---|---|
| Parameter | Treatment | Week | TxW | Location | Pen(LxT) | PenxW(LxT) | AR(I) | Residual |
| Day of Sample | 0.21 | <0.01 | 0.75 | 0.26 | — | <0.01 | <0.01 | <0.01 |
| Milk Yield, lb | | | | | | | | |
| Protein, lb | 0.25 | <0.01 | 0.19 | 0.25 | 0.11 | <0.01 | <0.01 | <0.01 |
| Fat, lb | 0.07 | <0.01 | 0.63 | 0.28 | 0.15 | 0.03 | <0.01 | <0.01 |
| Lactose, lb | 0.18 | <0.01 | 0.55 | 0.25 | 0.39 | <0.01 | <0.01 | <0.01 |
| SCM, lb | 0.06 | <0.01 | 0.61 | 0.25 | 0.23 | 0.01 | <0.01 | <0.01 |
| ECM, lb | 0.06 | <0.01 | 0.70 | 0.25 | 0.24 | <0.01 | <0.01 | <0.01 |
| FCM, lb | 0.05 | <0.01 | 0.72 | 0.25 | 0.22 | 0.01 | <0.01 | <0.01 |
| Milk:Feed | 0.06 | <0.01 | 0.78 | 0.27 | N/A | N/A | <0.01 | <0.01 |
| SCM:Feed | <0.01 | <0.01 | 0.96 | 0.24 | N/A | N/A | <0.01 | <0.01 |
| ECM:Feed | <0.01 | <0.01 | 0.95 | 0.25 | N/A | N/A | <0.01 | <0.01 |
| FCM:Feed | <0.01 | <0.01 | 0.90 | 0.24 | N/A | N/A | <0.01 | <0.01 |
| NE_{L} | 0.22 | <0.01 | 0.71 | — | N/A | N/A | 0.09 | <0.01 |

^{ab}Within a row, means without a common superscript differ (P < 0.05).
^{xy}Within a row, means without a common superscript differ (0.05 ≤ P ≤ 0.10).

TABLE 5
Dry matter intake, body weight, body condition score, milk yield and component
proportions and milk fatty acid results during experimental weeks 4 through
9 for lactating dairy cattle fed varying concentrations of monensin.

| | Sample | Treatment | | | | | Contrasts | | P-Values |
|---|---|---|---|---|---|---|---|---|---|
| Parameter | Size | 0 | 11 | 14.5 | 18 | SEM | Linear | Quad | Covariate |
| DMI | 36 | 59.9 | 59.5 | 59.5 | 60.2 | 0.57 | 0.87 | 0.97 | <0.01 |
| Milk Production Results | | | | | | | | | |
| Protein, % | 412 | 3.31 | 3.33 | 3.33 | 3.32 | 0.02 | 0.43 | 0.80 | <0.01 |
| Fat, % | 410 | 4.37 | 4.46 | 4.50 | 4.43 | 0.06 | 0.06 | 0.99 | <0.01 |
| Lactose, % | 411 | 4.64 | 4.65 | 4.66 | 4.66 | 0.03 | 0.22 | 0.66 | <0.01 |
| MUN, mg/dL | 407 | 9.9^{a} | 11.1^{by} | 11.0^{b} | 10.4^{abx} | 0.19 | 0.00 | 0.05 | <0.01 |
| Milk Fatty Acid Results | | | | | | | | | |
| De novo, g/100 g | 413 | 1.05^{a} | 1.09^{b} | 1.09^{b} | 1.07^{ab} | 0.06 | 0.01 | 0.35 | <0.01 |
| Mixed, g/100 g | 414 | 1.75^{a} | 1.79^{ab} | 1.82^{b} | 1.78^{ab} | 0.03 | 0.02 | 0.74 | <0.01 |
| Performed, g/100 g | 412 | 1.34 | 1.33 | 1.32 | 1.32 | 0.01 | 0.28 | 0.94 | <0.01 |
| Total FA, g/100 g | 411 | 4.14 | 4.21 | 4.23 | 4.16 | 0.08 | 0.16 | 0.91 | <0.01 |
| De novo, Relative | 411 | 25.32^{a} | 25.90^{c} | 27.78^{bc} | 25.45^{ab} | 0.86 | 0.02 | 0.10 | <0.01 |
| Mixed, Relative | 416 | 42.06 | 42.49 | 42.81 | 42.45 | 0.26 | 0.07 | 0.86 | <0.01 |
| Preformed, Relative | 415 | 32.56^{b} | 31.56^{a} | 31.39^{a} | 32.09^{ab} | 0.84 | 0.01 | 0.56 | <0.01 |

| | P-Values | | | Random Effect | | | | |
|---|---|---|---|---|---|---|---|---|
| Parameter | Treatment | Week | TxW | Location | Pen(LxT) | PenxW(LxT) | AR(I) | Residual |
| DMI | 0.74 | <0.01 | 1.00 | N/A | N/A | N/A | <0.01 | <0.01 |
| Milk Production Results | | | | | | | | |
| Protein, % | 0.82 | <0.01 | 0.12 | — | 0.11 | <0.01 | <0.01 | <0.01 |
| Fat, % | 0.21 | <0.01 | 0.67 | 0.30 | 0.25 | 0.06 | <0.01 | <0.01 |
| Lactose, % | 0.62 | <0.01 | 0.26 | 0.25 | 0.47 | <0.01 | <0.01 | <0.01 |
| MUN, mg/dL | <0.01 | <0.01 | 0.05 | — | 0.39 | <0.01 | <0.01 | <0.01 |
| Milk Fatty Acid Results | | | | | | | | |
| De novo, g/100 g | 0.01 | 0.04 | 0.97 | 0.24 | 0.48 | <0.01 | <0.01 | <0.01 |
| Mixed, g/100 g | 0.06 | <0.01 | 0.37 | 0.26 | 0.18 | — | <0.01 | <0.01 |
| Performed, g/100 g | 0.75 | <0.01 | 0.98 | — | — | <0.01 | <0.01 | <0.01 |
| Total FA, g/100 g | 0.28 | <0.01 | 0.82 | 0.25 | 0.33 | 0.03 | <0.01 | <0.01 |
| De novo, Relative | <0.01 | <0.01 | 0.73 | 0.24 | 0.24 | <0.01 | <0.01 | <0.01 |
| Mixed, Relative | 0.28 | <0.01 | 1.00 | — | — | <0.01 | <0.01 | <0.01 |
| Preformed, Relative | 0.01 | <0.01 | 0.06 | 0.24 | 0.03 | — | <0.01 | <0.01 |

^{abc}Within a row, means without a common superscript differ (P < 0.05).
^{xy}Within a row, means without a common superscript differ (0.05 ≤ P ≤ 0.10).

TABLE 6
Weekly averaged milk and component yield, corrected milk yield, feed efficiency
and net energy of lactation changes during experimental weeks 4 through
9 for lactating dairy cattle fed varying concentrations of monensin.

| | Sample | Treatment | | | | | Contrasts | | P-Values |
|---|---|---|---|---|---|---|---|---|---|
| Parameter | Size | 0 | 11 | 14.5 | 18 | SEM | Linear | Quad | Covariate |
| Milk Yield, lb | 419 | 88.6 | 90.4 | 89.8 | 89.4 | 0.74 | 0.27 | 0.34 | <0.01 |
| Protein, lb | 412 | 2.91 | 2.99 | 2.97 | 2.94 | 0.03 | 0.19 | 0.31 | <0.01 |
| Fat, lb | 411 | 3.84^{a} | 3.97^{ab} | 4.00^{b} | 3.91^{ab} | 0.04 | 0.02 | 0.70 | <0.01 |
| Lactose, lb | 413 | 4.10 | 4.19 | 4.19 | 4.16 | 0.04 | 0.11 | 0.52 | <0.01 |
| SCM, lb | 410 | 90.6^{ax} | 93.5^{aby} | 93.7^{b} | 92.3^{ab} | 0.79 | 0.02 | 0.44 | <0.01 |
| ECM, lb | 411 | 98.6^{x} | 101.6^{y} | 101.7^{y} | 100.2^{xy} | 0.85 | 0.03 | 0.40 | <0.01 |
| FCM, lb | 412 | 93.0^{x} | 95.9^{y} | 96.1^{y} | 94.6^{xy} | 0.81 | 0.03 | 0.47 | <0.01 |
| Milk:Feed | 35 | 1.52 | 1.55 | 1.54 | 1.50 | 0.02 | 0.87 | 0.96 | <0.01 |
| SCM:Feed | 35 | 1.53^{a} | 1.58^{b} | 1.58^{b} | 1.53^{a} | 0.01 | 0.03 | 0.74 | <0.01 |
| ECM:Feed | 35 | 1.66^{a} | 1.72^{b} | 1.72^{b} | 1.66^{a} | 0.01 | 0.05 | 0.69 | <0.01 |
| FCM:Feed | 35 | 1.57^{a} | 1.62^{b} | 1.63^{b} | 1.56^{a} | 0.01 | 0.02 | 0.62 | <0.01 |

| | P-Values | | | Random Effect | | | | |
|---|---|---|---|---|---|---|---|---|
| Parameter | Treatment | Week | TxW | Location | Pen(LxT) | PenxW(LxT) | AR(I) | Residual |
| Milk Yield, lb | 0.39 | <0.01 | 0.95 | — | 0.33 | <0.01 | <0.01 | <0.01 |
| Protein, lb | 0.26 | <0.01 | 0.07 | — | 0.15 | 0.02 | <0.01 | <0.01 |
| Fat, lb | 0.05 | <0.01 | 0.78 | — | 0.27 | 0.08 | <0.01 | <0.01 |
| Lactose, lb | 0.30 | <0.01 | 0.59 | — | — | 0.01 | <0.01 | <0.01 |
| SCM, lb | 0.05 | <0.01 | 0.68 | — | 0.33 | 0.02 | <0.01 | <0.01 |
| ECM, lb | 0.06 | <0.01 | 0.84 | — | 0.24 | 0.02 | <0.01 | <0.01 |
| FCM, lb | 0.06 | <0.01 | 0.87 | — | 0.28 | 0.02 | <0.01 | <0.01 |
| Milk:Feed | 0.21 | <0.01 | 0.97 | — | N/A | N/A | <0.01 | <0.01 |
| SCM:Feed | <0.01 | <0.01 | 0.95 | 0.35 | N/A | N/A | <0.01 | <0.01 |
| ECM:Feed | <0.01 | <0.01 | 0.90 | 0.49 | N/A | N/A | <0.01 | <0.01 |
| FCM:Feed | <0.01 | <0.01 | 0.93 | — | N/A | N/A | <0.01 | <0.01 |

^{ab}Within a row, means without a common superscript differ (P < 0.05).
^{xy}Within a row, means without a common superscript differ (0.05 ≤ P ≤ 0.10).

TABLE 7
Sample day milk and component yield, corrected milk yield, feed efficiency
and net energy of lactation changes during experimental weeks 4 through
9 for lactating dairy cattle fed varying concentrations of monensin.

| | Sample | Treatment | | | | | Contrasts | | P-Values |
|---|---|---|---|---|---|---|---|---|---|
| Parameter | Size | 0 | 11 | 14.5 | 18 | SEM | Linear | Quad | Covariate |
| Day of Sample | 412 | 88.9 | 90.6 | 91.3 | 90.6 | 1.76 | 0.03 | 0.80 | <0.01 |
| Milk Yield, lb | | | | | | | | | |
| Protein, lb | 406 | 2.93 | 3.01 | 3.02 | 2.97 | 0.07 | 0.09 | 0.71 | <0.01 |
| Fat, lb | 407 | 3.86^{a} | 3.98^{ab} | 4.06^{b} | 3.96^{ab} | 0.07 | 0.01 | 0.89 | <0.01 |
| Lactose, lb | 407 | 4.13 | 4.23 | 4.27 | 4.22 | 0.12 | 0.03 | 0.92 | <0.01 |
| SCM, lb | 406 | 91.3^{a} | 93.9^{ab} | 95.2^{b} | 93.4^{ab} | 2.12 | 0.01 | 0.96 | <0.01 |
| ECM, lb | 406 | 99.2^{a} | 102.0^{ab} | 103.3^{b} | 101.4^{ab} | 2.14 | 0.01 | 0.90 | <0.01 |
| FCM, lb | 407 | 93.5^{a} | 96.2^{ab} | 97.6^{b} | 95.8^{ab} | 2.01 | 0.01 | 0.96 | <0.01 |
| Milk:Feed | 35 | 1.52^{xy} | 1.54^{xy} | 1.57^{y} | 1.51^{x} | 0.03 | 0.17 | 0.29 | <0.01 |
| SCM:Feed | 35 | 1.54^{a} | 1.57^{abx} | 1.62^{by} | 1.54^{a} | 0.03 | <0.01 | 0.12 | <0.01 |
| ECM:Feed | 35 | 1.66^{a} | 1.71^{abx} | 1.75^{by} | 1.67^{a} | 0.04 | <0.01 | 0.24 | <0.01 |
| FCM:Feed | 35 | 1.57^{a} | 1.61^{b} | 1.66^{c} | 1.58^{ab} | 0.03 | <0.01 | 0.15 | <0.01 |

| | P-Values | | | Random Effect | | | | |
|---|---|---|---|---|---|---|---|---|
| Parameter | Treatment | Week | TxW | Location | Pen(LxT) | PenxW(LxT) | AR(I) | Residual |
| Day of Sample | 0.17 | <0.01 | 0.92 | 0.25 | — | <0.01 | <0.01 | <0.01 |
| Milk Yield, lb | | | | | | | | |
| Protein, lb | 0.21 | <0.01 | 0.21 | 0.25 | 0.20 | <0.01 | <0.01 | <0.01 |
| Fat, lb | 0.04 | <0.01 | 0.67 | 0.27 | 0.34 | 0.02 | <0.01 | <0.01 |
| Lactose, lb | 0.17 | <0.01 | 0.54 | 0.25 | — | <0.01 | <0.01 | <0.01 |
| SCM, lb | 0.04 | <0.01 | 0.71 | 0.25 | 0.41 | 0.01 | <0.01 | <0.01 |
| ECM, lb | 0.04 | <0.01 | 0.80 | 0.25 | 0.41 | <0.01 | <0.01 | <0.01 |
| FCM, lb | 0.03 | <0.01 | 0.81 | 0.25 | 0.41 | 0.01 | <0.01 | <0.01 |
| Milk:Feed | 0.08 | <0.01 | 0.90 | 0.28 | N/A | N/A | <0.01 | <0.01 |
| SCM:Feed | <0.01 | <0.01 | 0.98 | 0.25 | N/A | N/A | 0.03 | <0.01 |
| ECM:Feed | <0.01 | <0.01 | 0.97 | 0.25 | N/A | N/A | 0.03 | <0.01 |
| FCM:Feed | <0.01 | <0.01 | 0.96 | 0.25 | N/A | N/A | 0.01 | <0.01 |

^{abc}Within a row, means without a common superscript differ (P < 0.05).
^{xy}Within a row, means without a common superscript differ (0.05 ≤ P ≤ 0.10).

What is claimed is:

1. A method of increasing a milk production efficiency in a ruminant, comprising: providing a dietary formulation to the ruminant, wherein the dietary formulation comprises a feed composition and an amount of monensin selected from a range of about 11 g/ton to about 16 g/ton of the feed composition; and wherein milk production efficiency in the ruminant is increased.

2. The method of claim 1, wherein the ruminant is selected from the group consisting of cattle, buffalo, sheep, and goats.

3. The method of claim 1, wherein the ruminant is a cow.

4. The method of claim 1, wherein the increase is a statistically significant increase in milk production efficiency.

5. The method of claim 1, wherein the amount of monensin is selected from a range of about 11 g/ton to about 14.5 g/ton of the feed composition.

6. The method of claim 1, wherein the amount of monensin is about 11 g/ton of the feed composition.

7. The method of claim 1, wherein the amount of monensin is about 14.5 g/ton of the feed composition.

8. A method of improving a fatty acid profile in a ruminant, comprising: providing a dietary formulation to the ruminant, wherein the dietary formulation comprises a feed composition and an amount of monensin selected from a range of about 11 g/ton to about 16 g/ton of the feed composition.

9. The method of claim 8, wherein the ruminant is selected from the group consisting of cattle, buffalo, sheep, and goats.

10. The method of claim 8, wherein de novo fatty acids in the milk of the ruminant is increased.

11. The method of claim 8, wherein mixed fatty acids in the milk of the ruminant is increased.

12. The method of claim 8, wherein the amount of monensin is selected from a range of about 11 g/ton to about 14.5 g/ton of the feed composition.

13. The method of claim 8, wherein the amount of monensin is about 11 g/ton of the feed composition.

14. The method of claim 8, wherein the amount of monensin is about 14.5 g/ton of the feed composition.

15. A method of increasing a feed efficiency (ECM/DMI) in a ruminant, comprising providing a dietary formulation to the ruminant, wherein the dietary formulation comprises a feed composition and an amount of monensin selected from a range of about 11 g/ton to about 16 g/ton of the feed composition.

16. The method of claim 15, wherein the ruminant is selected from the group consisting of cattle, buffalo, sheep, and goats.

17. The method of claim 15, wherein the amount of monensin is selected from a range of about 11 g/ton to about 14.5 g/ton of the feed composition.

18. The method of claim 15, wherein the amount of monensin is about 11 g/ton of the feed composition.

19. The method of claim 15, wherein the amount of monensin is about 14.5 g/ton of the feed composition.

20. The method of claim 15, wherein the feed efficiency is increased by about 3.5% compared to a ruminant that does not receive dietary monensin.

21. The method of claim 15, wherein the feed efficiency is increased by about 3.0% compared to a ruminant that does not receive dietary monensin.
